SEQUENCE LISTING (2 sequences)

SEQ ID NO: 1 - protein, 381 aa, Homo sapiens
PLQKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 2 - DNA, 5855 bp, Homo sapiens
agttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctgcacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

(19) United States
(12) Patent Application Publication
(10) Pub. No.: US 2010/0137201 A1
(43) Pub. Date: Jun. 3, 2010

(54) MEDICAMENTS

(75) Inventors: Mark W. J. Ferguson, Manchester (GB); Hugh G. Laverty, Manchester (GB); Nicholas Occleston, Machester (GB); Sharon O'Kane, Manchester (GB); Kerry Nield, Manchester (GB); Nicholas Goldspink, Manchester (GB)

Correspondence Address: FOLEY AND LARDNER LLP;SUITE 500, 3000 K STREET NW, WASHINGTON, DC, 20007, US

(73) Assignee: RENOVO LIMITED

(21) Appl. No.: 12/306,501

(22) PCT Filed: Jun. 29, 2007
(86) PCT No.: PCT/GB07/02445

§ 371 Date: Dec. 23, 2008

(30) Foreign Application Priority Data
Jun. 30, 2006 (GB) 0613031.4

(51) Int. Cl. A61K 38/17; C07K 14/435

(52) U.S. Cl. 514/12; 530/350

(57) ABSTRACT

There is provided the use of WNT5A, or a therapeutically effective fragment or derivative thereof, in the preparation of a medicament for use in the prevention, reduction or inhibition of scarring. There is also provided the use of WNT5A, or a therapeutically effective fragment or derivative thereof, in the preparation of a medicament for use in the prevention and/or treatment of a fibrotic disorder. Further aspects relate to methods by which scarring may be prevented, reduced or inhibited, and by which fibrotic disorders may be prevented and/or treated. Therapeutically effective amounts of WNT5A, or its fragments or derivatives, that may be used in the medicaments or methods of the invention are also provided.

The present invention relates to medicaments for the prevention, reduction or inhibition of scarring. The invention also relates to medicaments for the prevention and/or treatment of fibrotic disorders. Furthermore, the invention provides methods for the prevention, reduction or inhibition of scarring, as well as methods for the prevention and/or treatment of fibrotic disorders.

The response to wounding is common throughout all adult mammals. The response is conserved between the majority of tissue types and in each case leads to the same result, formation of a scar. Many different processes are at work during the healing response, and much research has been conducted into discovering what mediates these processes, and how they interact with each other to produce the final outcome.

The healing response arises as the evolutionary solution to the biological imperative to prevent the death of a wounded animal. Thus, to overcome the risk of mortality due to infection or blood loss, the body reacts rapidly to repair the damaged area, rather than attempt to regenerate the damaged tissue.

A scar may be defined as the structure produced as a result of the reparative response. Since the injured tissue is not regenerated to attain the same tissue architecture present before wounding a scar may be identified by virtue of its abnormal morphology as compared to unwounded tissue. Scars are composed of connective tissue deposited during the healing process. A scar may comprise connective tissue that has an abnormal organisation (as seen in scars of the skin) and/or connective tissue that is present in an abnormally increased amount (as seen in scars of the central nervous system). Most scars consist of both abnormally organised and excess connective tissue.

The abnormal structure of scars may be observed with reference to both their internal structure (which may be determined by means of microscopic analysis) and their external appearance (which may be assessed macroscopically).

Extracellular matrix (ECM) molecules comprise the major structural component of both unwounded and scarred skin. In unwounded skin these molecules form fibres that have a characteristic random arrangement that is commonly referred to as a “basket-weave”. In general the fibres observed within unwounded skin are of larger diameter than those seen in scars. Fibres in scars also exhibit a marked degree of alignment with each other as compared to the random arrangement of fibres in unwounded skin. Both the size and arrangement of ECM may contribute to scars' altered mechanical properties, most notably increased stiffness, when compared with normal unwounded skin.

Viewed macroscopically, scars may be depressed below the surface of the surrounding tissue, or elevated above the surface of the undamaged skin. Scars may be relatively darker coloured than the unwounded tissue (hyperpigmentation) or may have a paler colour (hypopigmentation) than their surroundings. Either hyperpigmented or hypopigmented scars constitute a readily apparent cosmetic defect. Equally, scars may be redder than the surrounding skin, causing them to be noticeable and cosmetically unacceptable. It has been shown that the cosmetic appearance of a scar is one of the major factors contributing to the psychological impact of wounds and scars upon the sufferer, and that these effects can remain long after the wound itself has healed.

Scars may also have deleterious physical effects upon the sufferer. These effects typically arise as a result of the mechanical differences between scars and unwounded skin. The abnormal structure and composition of scars mean that they are typically less flexible than normal skin. As a result scars may be responsible for impairment of normal function (such as in the case of scars covering joints which may restrict the possible range of movement) and may retard normal growth if present from an early age.

Scarring may also occur at many other body sites, and the effects of scarring at these sites may also be deleterious to the sufferer. For example, scarring in the eye (whether as a result of accidental injury or surgical intervention) can impair vision and even lead to blindness. Scarring of the internal organs may lead to the formation of strictures and adhesions that significantly or totally impair function of the organ in question. Scarring of tendons and ligaments may cause lasting damage to these organs, and thereby reduce the motility or function of associated joints. Scarring associated with blood vessels, and particularly the valves of the heart, may occur after injury or surgery. The scarring may lead to restenosis, which causes a narrowing of the blood vessel and thus reduces the flow of blood through the scarred area. Scarring in the central or peripheral nervous system may prevent transmission along the nerve and may prevent or reduce reconnection of damaged nerve tissue.

The effects outlined above may all arise as a result of the normal progression of the wound healing response. There are, however, many ways in which this response may be abnormally altered; and these are frequently associated with even more damaging results.

One way in which the healing response may be altered is through the production of abnormal excessive scarring. Hypertrophic scars represent a severe form of scarring, and have marked adverse effects on the sufferer. Hypertrophic scars are elevated above the normal surface of the skin and contain excessive collagen arranged in an abnormal pattern. As a result such scars are often associated with a marked loss of normal mechanical function. This may be exacerbated by the tendency of hypertrophic scars to undergo contraction after their formation, an activity normally ascribed to their abnormal expression of muscle-related proteins (particularly smooth-muscle actin). Children suffer from an increased likelihood of hypertrophic scar formation, particularly as a result of burn injuries.

Keloids are another common form of pathological scarring. Keloid scars are not only elevated above the surface of the skin but also extend beyond the boundaries of the original injury. Keloids contain excessive connective tissue that is organised in an abnormal fashion, normally manifested as whorls of collagenous tissue. The causes of keloid formation are open to conjecture, but it is generally recognised that some individuals have a genetic predisposition to their formation. Both hypertrophic scars and keloids are particularly common in Afro-Caribbean and Mongoloid races.

A further common form of pathological scarring is pterygium in which a wedge-shaped fibrotic outgrowth of subconjunctival tissue may grow to the border of the cornea or beyond. Pterygium is more frequent among those frequently exposed to strong sunlight or dusty conditions.

Although scarring may be defined as the production of the structure that remains on healing of a wound, similar disturbances of the extracellular matrix are also associated with a number of medical conditions known as fibrotic disorders. In these disorders excessive fibrosis leads to pathological derangement and malfunctioning of tissue. Fibrotic disorders are characterised by the accumulation of fibrous tissue (predominately collagens, as found in scars) in an abnormal fashion within the damaged tissue. Accumulation of such fibrous tissues may result from a variety of disease processes, all of which lead to the same end result.

Fibrotic disorders are usually chronic. Examples of fibrotic disorders include cirrhosis of the liver, liver fibrosis, glomerulonephritis, pulmonary fibrosis, chronic obstructive pulmonary disease, scleroderma, myocardial fibrosis, fibrosis following myocardial infarction, central nervous system fibrosis following a stroke, neuro-degenerative disorders (e.g. Alzheimer's Disease, multiple sclerosis), proliferative vitreoretinopathy (PVR), arthritis, adhesions e.g. in the digestive tract, abdomen, pelvis, spine.

If not treated the pathological effects of fibrotic disorders may lead to organ failure, and ultimately to death.

The skilled person will appreciate that many of the mechanisms underlying the fibrotic response observed in fibrotic disorders are shared with the wound healing response which leads to scar formation. It may be expected that methods and medicaments that may be used to prevent or reduce fibrosis in one condition may also be of utility in the other.

Whilst much of the present specification concentrates primarily on the effects of wound healing or fibrotic disorders in man, it will be appreciated that many aspects of wound healing and the fibrotic response are conserved between most species of animals. Thus, the problems outlined above are also applicable to non-human animals, and particularly veterinary or domestic animals (e.g. horses, cattle, dogs, cats etc). By way of example, it is well known that adhesions resulting from the inappropriate healing of abdominal wounds constitute a major reason for the veterinary destruction of horses (particularly race horses). Similarly the tendons and ligaments of domestic or veterinary animals are also frequently subject to injury, and healing of these injuries may also lead to scarring associated with increased animal mortality.

Although the ill effects of normal and aberrant wound healing and of fibrotic disorders are well known there remains a lack of effective therapies able to reduce their effects. In the light of this absence it must be recognised that there exists a strongly felt need to provide treatments and medicaments that are able to prevent, reduce or inhibit scar formation, and to prevent and/or treat fibrotic disorders.

The WNT family of genes (wingless-type MMTV integration site family) encode a number of proteins that function as pleiotropic cell signalling molecules. These proteins, designated WNTs, share a number of conserved residues, including a characteristic cysteine pattern. It is these structural features, rather than shared function, that define the WNT proteins, since the effects of various WNT family members may differ markedly depending on the responding cells.

It is generally believed that Frizzled (Fz) molecules constitute the primary group of receptors for WNT family members. Frizzled receptors comprise seven membrane-spanning portions as well as a long amino terminal region designated the cysteine-rich domain (CRD). The CRD appears to constitute the WNT-binding portion of Fz receptors. Effective WNT signalling requires not only the presence of WNT and a Fz receptor, but also the presence of a protein of the LRP (LDL receptor related protein) class.

WNT5A is a member of the WNT family of signalling molecules. Human WNT5A is a 381 amino acid polypeptide, the sequence of which is shown in Sequence ID No. 1. The human and murine forms of WNT5A share 97% amino acid identity. The sequence of the gene encoding human WNT5A (also designated WNT5A) is set out in Sequence ID No. 2.

It is an aim of certain aspects of the present invention to provide medicaments suitable for the prevention and/or reduction and/or inhibition of scarring. It is an aim of further aspects of the present invention to provide methods of treatment suitable for use in the prevention, and/or reduction, and/or inhibition of scarring. It is an aim of certain embodiments of the invention to provide medicaments suitable for the prevention and/or treatment of fibrotic disorders. It is an aim of further embodiments of the invention to provide methods of treatment suitable for use in the prevention and/or treatment of fibrotic disorders. The medicaments and/or methods of the invention may constitute alternatives to those provided by the prior art, however, it is preferred that medicaments and/or methods of treatment provided by the invention may constitute improvements over the prior art.

According to a first aspect of the present invention there is provided the use of WNT5A, or a therapeutically effective fragment or derivative thereof, in the preparation of a medicament for use in the prevention, reduction or inhibition of scarring.

In a second aspect of the invention there is provided a method of preventing, reducing or inhibiting scarring, the method comprising administering a therapeutically effective amount of WNT5A, or a therapeutically effective fragment or derivative thereof, to a patient in need of such prevention, reduction or inhibition.

The present invention is based on the inventors' new and surprising finding that WNT5A, or therapeutically effective fragments or derivatives thereof, may be used in the prevention, reduction or inhibition of scarring. This prevention, reduction or inhibition of scarring can be effected at any body site and in any tissue or organ.

The inventors' findings are particularly surprising in the light of prior art reports that have suggested that induction of WNT5A expression by infection using genetically engineered retroviruses has no effect on scarring, since it does not effect the amount or orientation of extracellular matrix molecules deposited in the dermis during wound healing. Instead, previous reports have suggested that WNT5A expression only influences the epidermis and epidermal appendages. Previous research has indicated that the structure of the epidermis does not significantly influence the appearance of scars as assessed clinically (Beausang et al., 1998).

The WNT5A, or therapeutically effective fragment or derivative thereof, may preferably be administered to a patient's wound that would otherwise be likely to give rise to a scar.

Examples of specific contexts in which the prevention, reduction or inhibition of scarring that may be achieved using the medicaments and methods of the invention may be of benefit include, but are not limited to those selected from the group consisting of: use in the skin; use in the eye (including the prevention, reduction or inhibition of scarring resulting from eye surgery such as LASIK or PRK surgery); use in blood vessels; use in the peripheral or central nervous system (where prevention, reduction or inhibition of scarring may enhance neuronal reconnection); use in tendons, ligaments or muscle; use in the oral cavity, including the lips and palate (such as in preventing, reducing or inhibiting scarring resulting from treatment of cleft lip or palate); use in the internal organs such as the liver, heart, brain, digestive tissues and reproductive tissues; and use in body cavities such as the abdominal cavity, pelvic cavity and thoracic cavity (where prevention, reduction or inhibition of scarring may reduce the number of incidences of adhesion formation and/or the size of adhesions formed). The medicaments and methods of the invention may be used to prevent, reduce or inhibit adhesions, such as those occurring in the abdomen, pelvis or spine. It is particularly preferred that the medicaments and methods of the invention be used to prevent, reduce or inhibit scarring of the skin (dermal scarring).

WNT5A, or therapeutically effective fragments or derivatives thereof, may also be used in the prevention and/or treatment of fibrotic disorders. Thus, according to a third aspect of the present invention, there is provided the use of WNT5A, or a therapeutically effective fragment or derivative thereof, in the preparation of a medicament for use in the prevention and/or treatment of a fibrotic disorder.

In a fourth aspect of the invention there is provided a method of preventing and/or treating a fibrotic disorder, the method comprising administering a therapeutically effective amount of WNT5A, or a therapeutically effective fragment or derivative thereof, to a patient in need of such prevention and/or treatment.

Preferred fibrotic disorders that may be prevented and/or treated using medicaments or methods of the invention may be selected from the group consisting of: skin fibrosis; scleroderma; progressive systemic fibrosis; lung fibrosis; muscle fibrosis; kidney fibrosis; glomerulosclerosis; glomerulonephritis; uterine fibrosis; renal fibrosis; cirrhosis of the liver, liver fibrosis; chronic obstructive pulmonary disease; fibrosis following myocardial infarction; central nervous system fibrosis, such as fibrosis following stroke; fibrosis associated with neuro-degenerative disorders such as Alzheimer's Disease or multiple sclerosis; fibrosis associated with proliferative vitreoretinopathy (PVR); restenosis; endometriosis; ischemic disease and radiation fibrosis.

Except where the context requires otherwise, references to “medicaments of the invention” should be taken as referring to medicaments prepared in accordance with the first, third, fifth, sixth and seventh aspects of the invention. Medicaments of the invention comprise a pharmaceutically acceptable excipient, diluent or carrier in addition to the WNT5A, fragment or derivative. Medicaments of the invention may preferably be in the form of an injectable solution comprising WNT5A, or a therapeutically effective fragment or derivative thereof. Solutions suitable for localised injection (such as intradermal injection) constitute particularly preferred forms of the medicaments of the invention.

For the purposes of the present invention, a “therapeutically effective fragment or derivative” of WNT5A is considered to be any fragment or derivative of WNT5A that is capable of:
i) preventing, reducing or inhibiting scar formation; and/or
ii) preventing and/or treating a fibrotic disorder.

Preferably a therapeutically effective fragment or derivative of WNT5A may be one that is capable of preventing, reducing or inhibiting scar formation occurring at a wound to which the fragment or derivative of WNT5A is added; or capable of preventing the development of a fibrotic disorder at a site where the fragment or derivative is added; or capable of treating a fibrotic disorder at a site to which the fragment or derivative is added.

WNT proteins are generally palmitoylated on a cysteine residue. Studies in which palmitoylation of WNTs has been disrupted by acyl protein thioesterase indicate that the presence of palmitate is essential in order for WNTs to exert their biological activity.

The inventors believe that WNT5A is palmitoylated on the cysteine residue located at position 105 in the amino acid sequence shown in Sequence ID No. 1. Accordingly, the skilled person will appreciate that it is preferred that fragments of WNT5A for use in accordance with the invention should be fragments that comprise the cysteine residue located at position 105 of Sequence ID No. 1 (the skilled person will readily appreciate that the numbered position of this cysteine residue within a particular fragment may change depending on the length of the fragment in question). Preferred fragments of WNT5A may be palmitoylated fragments, and particularly those palmitoylated at cysteine 105.

Therapeutically effective fragments of WNT5A suitable for use in accordance with the present invention may comprise up to 10 contiguous amino acid residues from Sequence ID No. 1, preferably up to 100 contiguous amino acid residues, more preferably up to 200 contiguous amino acid residues, and even more preferably up to 300 contiguous amino acid residues. Fragments suitable for use in the medicaments and methods of the present invention include those comprising up to 380 amino acids residues of Sequence ID No. 1. Preferred fragments will comprise at least 10 contiguous amino acid residues from Sequence ID No. 1.

Preferred fragments may include amino acid residues involved in binding of WNT5A to its cellular receptors. Previous reports indicate that WNT5A is able to signal through a number of receptors, or receptor complexes. For example, it has been suggested that Fz5 acts as a receptor for WNT5A, and also that WNT5A is capable of inducing signalling via a Fz4/LRP5 complex, or an orphan tyrosine kinase receptor, Ror2 (He, et al. 1997; and Mikels and Nusse 2006).

It will be appreciated that it is the three dimensional structure of WNT5A that is important in considering receptor binding, and that accordingly suitable fragments may be selected based upon their ability to assume the requisite three dimensional conformation necessary for receptor binding.

Although peptides comprising all or part of WNT5A (as defined by Sequence ID No. 1) represent preferred agents for use in accordance with the present invention, it will be recognised that there are contexts in which the sensitivity of peptides to degradation may be disadvantageous. There are many known techniques by which peptide derivatives may be produced that have greater resistance to degradation than do the original peptides from which they are derived.

Peptoid derivatives may be expected to have greater resistance to degradation than do peptide agents of the invention, whilst retaining the same ability to inhibit scarring and/or to prevent fibrotic disorders. Suitable peptoid derivatives may be readily designed from knowledge of WNT5A's sequence and structure. Commercially available software may be used to develop suitable peptoid derivatives according to well-established protocols. It will be appreciated that the therapeutic effectiveness of peptoid and other derivatives may be investigated using the same techniques that allow assessment of therapeutic effectiveness of peptide fragments.

Retropeptoids based on WNT5A or its therapeutically effective fragments (but in which all amino acids are replaced by peptoid residues in reversed order) are also able to inhibit scarring and/or prevent fibrotic disorders. A retropeptoid may be expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

D-amino acid forms of the WNT5A or its therapeutically effective fragments also confer the requisite ability to inhibit scarring and/or to prevent fibrotic disorders. In the case of D-amino acid forms, the order of the amino acid residues comprising the derivative is reversed as compared to those in the original peptide. The preparation of derivatives using D-amino acids rather than L-amino acids greatly decreases any unwanted breakdown of such an agent by normal metabolic processes, decreasing the amounts of agent which need to be administered, along with the frequency of its administration.

It will be appreciated that derivatives suitable for use in the medicaments and methods of the invention clearly include both those derived from full length WNT5A and those derived from therapeutically effective fragments of WNT5A.

As mentioned above, human WNT5A (as set out in Sequence ID No. 1) represents a preferred WNT5A that may be used in the medicaments or methods of the invention. Therapeutically effective fragments or derivatives of human WNT5A represent preferred fragments or derivatives for use in accordance with the invention. WNT5A, or a therapeutically effective fragment or derivative thereof, for use in the medicaments or methods of the invention may be manufactured in any way known to the skilled artisan. In particular, it may be preferred that recombinant WNT5A, or therapeutically effective fragments or derivatives thereof, such as recombinant human WNT5A, or a therapeutically effective fragment or derivative thereof, be used in the medicaments or methods of the invention. Recombinant human proteins may advantageously be produced in prokaryotic host cells. Such production has the advantage that large quantities of the protein may be produced, that the protein may be readily purified, and that the protein may be produced in conditions that reduce the risk of contamination with potentially deleterious agents that may prohibit therapeutic use of the protein (such contamination being a notable problem with proteins produced from eukaryotic cells or cell cultures).

For the purposes of the present invention a “therapeutically effective amount” of WNT5A, or a fragment or derivative thereof, is considered to be an amount of WNT5A, or a fragment or derivative thereof, that is able to bring about a required:
i) prevention, reduction or inhibition of scarring; or
ii) prevention and/or treatment of a fibrotic disorder.

A therapeutically effective amount of WNT5A (or a fragment or derivative thereof) may be an amount that is effective to reduce scarring of a treated wound by at least 10% compared to scarring produced on healing of a comparable untreated wound. Preferably a therapeutically effective amount may be capable of achieving at least a 20% reduction in scarring, more preferably at least 50%, even more preferably at least 75% and most preferably at least a 90% reduction in scarring compared to scarring produced on healing of a non-treated wound.

A therapeutically effective amount of WNT5A, or a fragment or derivative thereof, may preferably be an amount able to therapeutically alter the abundance and/or orientation of ECM components (such as collagen) in a treated scar.

A medicament of the invention should provide a therapeutically effective amount of WNT5A, or a fragment or derivative thereof. Preferably a medicament of the invention may be provided in the form of a dosage unit, the dosage unit comprising a therapeutically effective amount of WNT5A, or a fragment or derivative thereof.

The skilled person will appreciate that a fragment or derivative of WNT5A that has little inherent therapeutic activity will still be therapeutically effective if administered in a quantity that provides a therapeutically effective amount.

The inventors have surprisingly found that when administered in too great a quantity WNT5A, or its therapeutically effective fragments or derivatives, serves to worsen scarring. It will be appreciated that an amount of WNT5A, or a therapeutically effective fragment or derivative thereof, that does not improve scarring, or serves to worsen scarring, will not constitute a therapeutically effective amount for the purposes of the present invention.

In general, a therapeutically effective amount of WNT5A (or a fragment or derivative thereof) to be applied to a wound, scar, or a site of a fibrotic disorder in order to prevent, reduce or inhibit scarring may be determined by a physician with reference to factors including the size of a wound, amount of fibrosis, and presence or absence of factors contributing to pronounced or pathological scarring. Such factors are considered in more detail elsewhere in the specification

The following paragraphs provide further guidance as to the selection of a suitable therapeutically effective amount of WNT5A, or a fragment or derivative thereof, for use in the medicaments or methods of the invention.

Generally, it may be preferred that a centimetre of wound, or cm² of wound or fibrotic disorder, will receive an amount of WNT5A (or a fragment or derivative thereof) less than 2000 ng (or approximately 44 picomoles) per 24 hours during a “course” of treatment (a course of treatment comprising one or more administrations of WNT5A, or a fragment or derivative thereof, by a medicament or method of the invention) to prevent, reduce or inhibit scarring.

Within a course of treatment to prevent, reduce or inhibit scarring, WNT5A, or a fragment or derivative thereof, may be provided in one, or more administrations per 24 hours. Thus, in the event that a single administration is to be used, a therapeutically effective amount of WNT5A, or a fragment or derivative thereof, to be administered to a centimetre of wound (or cm² of wound or fibrosis) should be less than 2000 ng (or approximately 44 picomoles) per administration. In the event that multiple administrations are to be used, the total amount of WNT5A, or a fragment or derivative thereof, to be administered to a centimetre of wound (or cm² of wound or fibrosis) over the total 24 hour period should be less than 2000 ng (or approximately 44 picomoles).

It will be appreciated that, in some cases, a course of treatment may not last longer than 24 hours (for example in the case that a patient is discharged from hospital or other medical supervision within a 24 hour period), and in such cases the total therapeutic amount of WNT5A (or a fragment or derivative thereof) administered per centimetre of wound, or cm2 of wound or fibrosis, may be less than 2000 ng (or approximately 44 picomoles).

In other cases, for example in the case of extensive wounding or scarring, or scarring or fibrotic disorders that demonstrate a resistance to treatment, it may be preferred that a course of treatment extends for a protracted period of time, and that the total amount of WNT5A, or a fragment derivative thereof, administered to a given centimetre of wound (or cm² of wound or fibrosis) during a course of treatment may be considerably larger than 2000 ng (indeed the total amount administered may be many times larger). However, in order to achieve effective prevention, reduction or inhibition of scarring, it may be preferred that the total amount of WNT5A, or a fragment or derivative thereof, to be administered to a centimetre of wound, or cm2 of wound or fibrosis, per 24 hours of this protracted treatment should be less than 2000 ng (or approximately 44 picomoles).

By way of further example, the inventors have established that a therapeutically effective amount of WNT5A, or a fragment or derivative thereof, should be less than 44 picomoles (pmoles) per linear cm (or cm²) of a wound the scarring of which it is wished to inhibit, or per cm² of a fibrotic disorder it is wished to treat (whether administered in a single incidence, or in a number of incidences over a period of 24 hours). By way of contrast, the provision of an amount of WNT5A up to about 33 pmoles per linear cm (or cm²) of a wound or fibrotic disorder will constitute a therapeutically effective amount.

By way of further illustration, the provision of between 1 ng and 1500 ng of WNT5A per linear cm of wound, or cm² of a wound or fibrotic disorder, over a 24 hour period will constitute a preferred therapeutically effective amount. Such a therapeutically effective amount may preferably be provided by two or more incidences of treatment within the 24 hour period.

The inventors have found that, a therapeutically effective amount of WNT5A should be less than 2000 ng per linear cm of wound, or cm² of a wound or fibrotic disorder, over a 24 hour period, and may preferably be less than 1600 ng.

Generally a therapeutic amount of WNT5A to be administered per linear cm of wound, or cm² of a wound or fibrotic disorder, over a 24 hour period should comprise more than 0.2 ng.

Preferred therapeutically effective amounts of WNT5A, its fragments or derivatives, may be investigated using in vitro and in vivo models, and suitable assessments of efficacy made with reference to various parameters for the measurement of scarring, as described elsewhere in the specification.

The invention provides the use of WNT5A, or a fragment or derivative thereof, in the manufacture of a medicament for administration in an amount to provide per cm² of the administered area a level of WNT5A activity equivalent to that provided by up to 2000 ng of WNT5A, for preventing, reducing or inhibiting scarring.

The invention also provides the use of WNT5A, a therapeutically effective fragment or derivative thereof, in the preparation of a medicament for administering between 0.01 and 35 picomoles of WNT5A, or the fragment or derivative thereof, per linear cm or cm² of a wound. Such a medicament may prevent, reduce or inhibit scarring. A medicament in accordance with this aspect of the invention may preferably provide between 0.02 picomoles and 35 picomoles, for example 0.04 picomoles, 0.44 picomoles, 4.4 picomoles, 22 picomoles, or 33 picomoles of the WNT5A, fragment or derivative.

In a further aspect the invention provides the use of WNT5A, a therapeutically effective fragment or derivative thereof, in the preparation of a medicament for administering between 0.01 and 35 picomoles of WNT5A, or the fragment or derivative thereof, per cm² of fibrosis associated with a fibrotic disorder. Such a medicament may prevent and/or treat the fibrotic disorder. A medicament in accordance with this aspect of the invention may preferably provide between 0.02 picomoles and 35 picomoles, for example 0.04 picomoles, 0.44 picomoles, 4.4 picomoles, 22 picomoles, or 33 picomoles of the WNT5A, fragment or derivative.

In a still further aspect, the invention provides the use of WNT5A, or a therapeutically effective fragment or derivative thereof, in the preparation of a medicament for the prevention, reduction or inhibition of scarring, wherein the administration pattern for administering the medicament comprises administering a therapeutically effective amount of WNT5A, or a fragment or derivative thereof, prior to formation of a wound, and administering a further therapeutically effective amount of WNT5A, or a fragment or derivative thereof, after formation of the wound.

Administration of the further therapeutically effective amount of WNT5A, or a fragment or derivative thereof, may preferably occur approximately 24 hours after formation of the wound.

It will be appreciated that the terms “per 24 hours” or “in a 24 hour period” as used in the present application are intended to provide guidance as to the amount of WNT5A, or a fragment or derivative thereof, that may be administered to a given site during a period of treatment while still providing a therapeutic effect. The inventors believe that the amounts specified in these terms may generally be provided over two consecutive days of treatment while still providing a therapeutically effective amount (though administered over a period that may be somewhat more than 24 hours).

In general, the uses described herein may further comprise the repetition of administration of therapeutically effective amounts of WNT5A, or a fragment or derivative thereof, for as long as necessary to achieve prevention, reduction or inhibition of scarring.

It is anticipated that the majority of fragments or derivatives of WNT5A, that share the biological activity of WNT5A, will also exhibit therapeutic activity that will make them suitable for use in the methods and medicaments of the invention.

“Biological activity” in the context of the present invention may be assessed in vitro or in vivo, but is preferably assessed in vivo. Accordingly, it will be recognised that for WNT5A, or fragments or derivatives thereof, to be deemed biologically active in accordance with the present invention it is not necessary for the WNT5A, fragment or derivative to exhibit biological activity measurable by both in vitro and in vivo means, but merely that the WNT5A, fragment or derivative is able to exhibit biological activity that can be measured either in vitro or in vivo, and preferably activity that can be measured in vivo.

Suitably, the biological activity of WNT5A, its fragments or derivatives, may be measured with reference to the ability of the WNT5A, or fragment or derivative thereof, to prevent, reduce or inhibit scarring.

For the sake of brevity, references in the present specification to “inhibition” of scarring should be taken, except where the context requires otherwise, to also encompass the prevention or reduction of scarring. Similarly, references to “treatment” of a fibrotic disorder, or disorders, should, except where the context requires otherwise, be taken also to encompass the prevention of fibrotic disorders.

The extent of inhibition of scarring or treatment of a fibrotic disorder, that may be required in order to achieve a therapeutic effect will be apparent to, and may readily be determined by, a clinician responsible for the care of the patient. The clinician may determine a suitable assessment of the extent of inhibition of scarring, or treatment of a fibrotic disorder, achieved using WNT5A or its therapeutically effective fragments or derivatives. Such an assessment may, for example, be made with reference to suggested methods of measurement described herein.

The extent to which inhibition of scarring or treatment of fibrotic disorders is achieved may be assessed with reference to the effects that arise in human patients treated with the methods or medicaments of the invention. Alternatively, inhibition of scarring, or treatment of fibrotic disorders, may be assessed with reference to experimental investigations using suitable in vitro or in vivo models. The use of experimental models to investigate inhibition of scarring or treatment of fibrotic disorders may be particularly preferred in assessing the therapeutic effectiveness of fragments or derivatives of WNT5A, or in establishing therapeutically effective amounts of WNT5A, its fragments or derivatives.

Animal models of scarring or fibrotic disorders represent preferred experimental models for in vivo assessment of the extent of scar inhibition or treatment of fibrotic disorders that may be achieved. Suitable models will be known to those skilled in the art. Examples of such models are described below for illustrative purposes.

Therapeutically effective fragments or derivatives of WNT5A, and therapeutically effective amounts of WNT5A, its fragments or derivatives thereof, may be selected with reference to any or all of the considerations described in the present specification.

Treatment of wounds with WNT5A, or its therapeutically effective fragments or derivatives, may result in the inhibition of scarring that may otherwise be expected to occur on healing of untreated wounds. The inventors believe that treatment in this manner may have an impact on both the macroscopic and microscopic appearance of scars formed from treated wounds; macroscopically the scars may be less noticeable and blend with the surrounding skin, microscopically the scars may exhibit an internal structure more akin to normal skin structure. In particular, microscopically a treated scar may exhibit an abundance and orientation of ECM molecules such as collagen that is more similar to that found in unwounded skin than that found in untreated scars.

For present purposes an “untreated wound” should be considered to be any wound that has not been exposed to a therapeutically effective amount of WNT5A, or a therapeutically effective fragment or derivative thereof. A “control-treated wound” will be an untreated wound to which a control substance has been administered.

In contrast, a “treated wound” may be considered to be a wound exposed to a therapeutically effective amount of WNT5A, or a therapeutically effective fragment or derivative thereof. Thus a treated wound may be a wound which has been provided with a medicament of the invention, or which has received treatment in accordance with the methods of the invention.

Inhibition of scarring, using the medicaments and methods of the invention, can be effected at any body site and in any tissue or organ so far investigated. For illustrative purposes the antiscarring activity of medicaments and methods of the invention will primarily be described with reference to inhibition of scarring that may be brought about in the skin (the body's largest organ). However, the skilled person will immediately appreciated that many of the factors that may be relevant when considering inhibition of scarring in the skin will also be relevant to inhibition of scarring in other organs or tissues.

In the skin, treatment may improve the macroscopic and microscopic appearance of scars; macroscopically the scars may be less visible and blend with the surrounding skin, microscopically the collagen fibres within the scar may have morphology and organisation that is more similar to those in the surrounding skin. The prevention, reduction or inhibition of scarring within the context of the present invention should be understood to encompass any degree of prevention, reduction or inhibition in scarring as compared to the level of scarring occurring in a control-treated or untreated wound (as defined above). Throughout the specification references to “prevention”, “reduction” or “inhibition” of scarring are generally to be taken, except where the context requires otherwise, to be equivalent mechanisms that are all manifested in anti-scarring activity.

The inhibition of scarring achieved using methods and medicaments of the invention may be assessed and/or measured with reference to either the microscopic or macroscopic appearance of a treated scar as compared to the appearance of an untreated scar. Inhibition of scarring may also suitably be assessed with reference to both macroscopic and microscopic appearance of a treated scar. It may generally be preferred that inhibition of scarring be assessed with reference to the microscopic structure of scars.

For the present purposes a “treated scar” may be defined as a scar formed on healing of a treated wound, whereas an “untreated scar” may be defined as the scar formed on healing of an untreated wound, or a wound treated with placebo or standard care (examples of control-treated wounds). Suitable comparison scars may preferably be matched to the treated scar with reference to scar age, site, size and patient.

In considering the macroscopic appearance of a scar resulting from a treated wound, the extent of scarring, and hence the magnitude of any inhibition of scarring achieved, may be assessed with reference to any of a number of parameters.

Suitable parameters for the macroscopic assessment of scars may include:
i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Similarly, scars may be redder than the surrounding skin. In this case inhibition of scarring may be demonstrated when the redness of a treated scar fades earlier, or more completely, or to resemble more closely the appearance of the surrounding skin, compared to an untreated scar.
ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar.
iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar.
iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out above. More preferably a treated scar may demonstrate inhibited scarring with reference to at least two of the parameters, even more preferably at least three of the parameters, and most preferably all four of these parameters. An over-all assessment of scarring may be made using, for example, a Visual Analogue Scale or a digital assessment scale.

Suitable parameters for the microscopic assessment of scars may include:
i) Thickness of extracellular matrix (ECM) fibres. Scars typically contain thinner ECM fibres than are found in the surrounding skin. This property is even more pronounced in the case of keloid and hypertrophic scars. Inhibition of scarring may be demonstrated when the thickness of ECM fibres in a treated scar more closely approximates the thickness of ECM fibres found in unscarred skin than does the thickness of fibres found in an untreated scar.
ii) Orientation of ECM fibres. ECM fibres found in scars tend to exhibit a greater degree of alignment with one another than do those found in unscarred skin (which have a random orientation frequently referred to as “basket weave”). The ECM of pathological scars such as keloids and hypertrophic scars may exhibit even more anomalous orientations, frequently forming large “swirls” or “capsules” of ECM molecules. Accordingly, inhibition of scarring may be demonstrated when the orientation of ECM fibres in a treated scar more closely approximates the orientation of ECM fibres found in unscarred skin than does the orientation of such fibres found in an untreated scar.
iii) ECM composition of the scar. The composition of ECM molecules present in scars shows differences from that found in normal skin, with a reduction in the amount of elastin present in ECM of scars. Thus inhibition of scarring may be demonstrated when the composition of ECM fibres in the dermis of a treated scar more closely approximates the composition of such fibres found in unscarred skin than does the composition found in an untreated scar.
iv) Cellularity of the scar. Scars tend to contain relatively fewer cells than does unscarred skin. It will therefore be appreciated that inhibition of scarring may be demonstrated when the cellularity of a treated scar more closely approximates the cellularity of unscarred skin than does the cellularity of an untreated scar.

It is surprising to note that the overall appearance of scars is little influenced by the epidermal covering of the scar, even though this is the part of the scar that is seen by the observer. Instead, the inventors find that the properties of the neo-dermis produced on resolution of the wound healing response have far greater impact on the perception of extent of scarring, as well as on the function of the scarred tissue. Accordingly assessments of criteria associated with the dermis, rather than epidermis, prove to be the most useful in determining inhibition of scarring.

The thickness of ECM fibres and orientation of ECM fibres may be favoured parameters, for assessing inhibition of scarring. A treated scar may preferably have improved ECM orientation (i.e. orientation that is more similar to unwounded skin than is the orientation in an untreated scar).

A treated scar will preferably demonstrate inhibition of scarring as assessed with reference to at least one of the parameters for microscopic assessment set out above. More preferably a treated scar may demonstrate inhibition of scarring with reference to at least two of the parameters, even more preferably at least three of the parameters, and most preferably all four of these parameters.

The inhibition of scarring occurring at a treated wound may further be assessed with reference to suitable parameters used in the:
i) macroscopic clinical assessment of scars, particularly the assessment of scars upon a subject;
ii) assessment of photographic images of scars;
iii) assessment of silicone moulds or positive plaster casts made from silicone moulds of scars; and
iv) microscopic assessment of scars, for example by histological analysis of the microscopic structure of scars.

It will be appreciated that inhibition of scarring of a treated wound may be indicated by improvement of one or more of such suitable parameters, and that in the case of inhibition as assessed with reference to a number of parameters that these parameters may be combined from different assessment schemes (e.g. inhibition as assessed with reference to at least one parameter used in macroscopic assessment and at least one parameter used in microscopic assessment).

Inhibition of scarring may be demonstrated by an improvement in one or more parameters indicating that a treated scar more closely approximates unscarred skin with reference to the selected parameter(s) than does an untreated or control scar.

Suitable parameters for the clinical measurement and assessment of scars may be selected based upon a variety of measures or assessments including those described by Beausang et al (1998) and van Zuijlen et al (2002).

Typically, suitable parameters may include:

1. Assessment with Regard to Visual Analogue Scale (VAS) Scar Score.

Prevention, reduction or inhibition of scarring may be demonstrated by a reduction in the VAS score of a treated scar when compared to a control scar. A suitable VAS for use in the assessment of scars may be based upon the method described by Beausang et al. (1998).

2. Assessment with Regard to a Categorical Scale.

Prevention, reduction or inhibition of scarring may be determined by allocating scars to different categories e.g. barely noticeable, blends well with normal skin, distinct from normal skin etc; or by comparing a treated or untreated scar and assigning any differences between them to categories e.g. mild, moderate, major etc. This assessment may be done by the patient, by the investigator, by an independent panel either on the patient or on photographs/moulds.

### 3. Scar Height, Scar Width, Scar Perimeter, Scar Area or Scar Volume.

The height and width of scars can be measured directly upon the subject, for example by use of manual measuring devices such as callipers. Scar width, perimeter and area may be measured either directly on the subject, by image analysis of photographs of the scar, or on plaster casts of impressions of the scar. The skilled person will also be aware of further non-invasive methods and devices that can be used to investigate suitable parameters, including silicone moulding, ultrasound, optical three-dimensional profilimetry and high resolution Magnetic Resonance Imaging.

Inhibition of scarring may be demonstrated by a reduction in the height, width, area, perimeter or volume, or any combination thereof, of a treated scar as compared to an untreated scar.

4. Appearance and/or Colour of Scar Compared to Surrounding Unscarred Skin.

The appearance or colour of a treated scar may be compared to that of surrounding unscarred skin, and the differences (if any) compared with the difference between the appearance and colour of untreated scars and unscarred skin. Such a comparison may be made on the basis of a visual assessment of the respective scars and unscarred skin. The appearance of a scar may be compared with unscarred skin with reference to whether the scar is lighter or darker, or redder, than the unscarred skin. The respective colours of the scars and skin may be perfectly matched to one another, slightly mismatched, obviously mismatched or grossly mismatched.

Alternatively or additionally to visual assessment, there are a number of non-invasive colourimetry devices which are able to provide data with respect to pigmentation of scars and unscarred skin, as well as redness of the skin (which may be an indicator of the degree of vascularity present in the scar or skin). Examples of such devices include the X-rite SP-62 spectrophotometer, Minolta Chronometer CR-200/300; Labscan 600; Dr. Lange Micro Colour; Derma Spectrometer; laser-Doppler flow meter; and Spectrophotometric intracutaneous Analysis (SIA) scope.

Inhibition of scarring may be demonstrated by a smaller magnitude of difference between the appearance or colour of treated scars and unscarred skin than between untreated scars and unscarred skin.

### 5. Scar Distortion and Mechanical Performance

Scar distortion may be assessed by visual comparison of a scar and unscarred skin. A suitable comparison may categorise a selected scar as causing no distortion, mild distortion, moderate distortion or severe distortion.

The mechanical performance of scars can be assessed using a number of non-invasive methods and devices based upon suction, pressure, torsion, tension and acoustics. Suitable examples of known devices capable of use in assessing mechanical performance of scars include Indentometer, Cutometer, Reviscometer, Visco-elastic skin analysis, Dermaflex, Durometer, Dermal Torque Meter, Elastometer.

Inhibition of scarring may be demonstrated by a reduction in distortion caused by treated scars as compared to that caused by untreated scars. It will also be appreciated that inhibition of scarring may be demonstrated by the mechanical performance of unscarred skin being more similar to that of treated scars than of untreated scars.

### 6. Scar Contour and Scar Texture

Scar contour may be investigated by means of visual assessment. Suitable parameters to consider in such an assessment include whether or not a scar is flush with surrounding skin, slightly proud, slightly indented, hypertrophic or keloid. The texture of a scar may be assessed with reference to the scar's appearance, and this may also be undertaken by a visual assessment as to whether the scar is, for instance, matt or shiny or has a roughened or smooth appearance as compared to unscarred skin.

Scar texture may additionally be assessed with reference to whether the scar has the same texture as unscarred skin (normal texture), is just palpable, firm or hard compared to unscarred skin. The texture of scars may also be assessed with reference to the Hamilton scale (described in Crowe et al, 1998).

In addition to the techniques set out above, there are a number of non-invasive profilimetry devices that use optical or mechanical methods for assessment of scar contour and/or texture. Such assessments may be carried out on the body of the subject or, for example, on silicone mould impressions of scars, or on positive casts made from such impressions.

Inhibition of scarring may be demonstrated in the event that treated scars have scar profiles and textures more comparable to unscarred skin than do untreated scars.

### Photographic Assessments

### Independent Lay Panel

Photographic assessment of treated and untreated scars may be performed by an independent lay panel of assessors using standardised and calibrated photographs of the scars. The scars may be assessed by an independent lay panel to provide categorical ranking data (e.g. that a given treated scar is “better”, “worse” or “no different” when compared to an untreated scar) and quantitative data using a Visual Analogue Scale (VAS) based upon the method described by Beausang et al. (1998). The capture of these data may make use of suitable software and/or electronic system(s) as described in the applicant's co-pending patent application.

### Expert Panel

Photographic assessment of treated and untreated scars may alternatively or additionally be performed by a panel of expert assessors using standardised and calibrated photographs of the scars to be assessed. The panel of experts may preferably consist of individuals skilled in the art, suitable examples of which include plastic surgeons and scientists having relevant technical backgrounds.

Such assessment may provide categorical data, as described above, or with respect to the comparison of a time-course of images of selected treated and untreated scars.

Suitable assessments that may be useful in assessing inhibition of scarring include:

Identification of the best scar within a series of scars. For the purposes of the present invention the best scar may be considered to be the one which most closely resembles the surrounding skin. Once the best scar has been identified the magnitude of the difference between scars may be considered, for example, whether the difference between scars is slight or obvious. Further parameters that may be considered include the earliest time after scar formation at which a difference between scars may be detected, the time post-formation at which the difference between scars is most obvious (or alternatively the finding that the difference continues after the last timepoint assessed), as well as considering whether or not the better scar remains consistently better.

Consideration may also be given to whether or not one scar is consistently redder than the other, and whether the redness fades over the time-points considered (or continues after the last timepoint) and if so at what time after scar formation. An expert panel may also consider at what time after formation any difference in redness becomes detectable, as well as the time post-formation at which the difference in redness is most obvious.

An expert panel may also consider whether or not one of a treated or untreated scar is consistently paler than the other, or paler than unscarred skin. In the event that a difference in paleness is detectable consideration may be given to the time after scar formation at which the difference may be detected, the time at which the difference is most obvious, and the time at which the difference disappears.

A further parameter that may be assessed by an expert panel is the texture of treated and untreated scars. In comparing treated and untreated scars the expert panel may consider which of the scars has the texture most similar to that of unscarred skin, the earliest time after scar formation at which any difference in texture may be detected, the time post formation at which any difference is most obvious, and the time at which any difference disappears

Comparison of treated and untreated scars may further assess which of the scars is narrowest, and which of the scars is shortest. Consideration may also be given to the shape of the scar and the proportion of the scar margin that is distinguishable from the surrounding skin. As with previously described visual assessments and assessments of colour, the presence, degree and location of hyper-pigmentation may also be considered.

### Clinical Assessment

A clinician: investigator or not, or an independent panel of clinicians may assess the scar(s) on a patient using any of the forgoing parameters e.g. VAS, colour, categorical scales etc.

### Patient Assessment

The patient may assess their own scars and/or compare scars by means of a structured questionnaire. This questionnaire may measure their satisfaction with the scar, how well the scar blends with the surrounding skin, as well as the effect of the scar on their daily life e.g. do they hide it with clothes, avoid exposing it; as well as scar symptoms e.g. itch, pain, paresthesia. Inhibition of scarring may be determined by the treated scar being rated better, causing the patient fewer problems, causing fewer and less scar symptoms, and with an increase in patient satisfaction compared to an untreated scar.

### Macroscopic Analysis

As noted above, one of the ways in which the quality of treated and untreated scars may be compared is by microscopic assessment. Microscopic assessment of scar quality may typically be carried out using histological sections of scars. The process of microscopically assessing and measuring scars may take into consideration categorical data based on the following suitable parameters:

1. Collagen organisation. In assessing collagen organisation reference may be made to the orientation of collagen fibres present in the scar, the density of such fibres and collagen fibre thickness in the papillary and reticular dermis. An inhibition of scarring may be indicated when a treated scar contains collagen organisation that more closely approximates that found in unwounded skin than does the organisation in untreated or control treated scars.
2. Abundance of ECM components. Scars typically contain an increased amount of ECM components such as collagen when compared to unwounded skin. An inhibition of scarring may be indicated when a treated scar contains reduced abundance of ECM components when compared to untreated or control treated scars.
3. Visual analogue scale (VAS) assessment of collagen organisation and abundance in the papillary dermis and the reticular dermis may also provide a useful index of scar quality.
4. Angiogenesis and Inflammation. Consideration may be given to the number of blood vessels present, the size of the blood vessels present and evidence of inflammation, including an assessment of any level of inflammation present. An inhibition of scarring may be indicated when a treated scar contains blood vessels and inflammatory cells in quantities and arrangements that more closely approximate those found in unwounded skin than those found in untreated or control treated scars.

Other features that may be taken into account in assessing the microscopic quality of scars include elevation or depression of the scar relative to the surrounding unscarred skin, and the prominence or visibility of the scar at the normal dermal interface.

It will be seen that the assessments described above allow the generation of scar ranking data which is able to provide an indication as to whether a treated scar is better, worse or no different compared to a control, untreated or other suitable comparator scar.

In addition to categorical data, quantitative data (preferably relating to the above parameters) can be generated using image analysis in combination with suitable visualisation techniques. Examples of suitable visualisation techniques that may be employed in assessing scar quality are specific histological stains or immuno-labelling, wherein the degree of staining or labelling present may be quantitatively determined by image analysis

Quantitative data may be usefully and readily produced in relation to the following parameters:
1. Scar width, height, elevation, volume and area.
2. Number, size, area (i.e. cross-section) and location of blood vessels.
3. Degree of inflammation, number, location and populations/types of inflammatory cells present.
4. Collagen organisation, collagen fibre thickness, collagen fibre density.
5. Number and orientation of fibroblasts.
6. Quantity and orientation of other ECM molecules e.g. elastin, fibronectin

Prevention, reduction or inhibition of scarring may be demonstrated by a change in any of the parameters considered above such that a treated scar more closely resembles unscarred skin than does a control or untreated scar (or other suitable comparator).

The assessments and parameters discussed above are suitable for assessment of the effects of WNT5A, or fragments or derivatives thereof, as compared to control, placebo or standard care treatment in animals or humans. Appropriate statistical tests may be used to analyse data sets generated from different treatments in order to investigate significance of results.

Preferably inhibition of scarring may be demonstrated with reference to more than one parameter. More preferably inhibition of scarring may be demonstrable with reference to both a clinical (i.e. observed on the subject) parameter and a photographic parameter. Even more preferably inhibition of scarring may be demonstrable with reference to a clinical parameter, a photographic parameter, and also a microscopic assessment parameter (for instance a histological parameter). Most preferably inhibition of scarring may be demonstrable with reference to a clinical VAS score, external lay panel VAS score and ranking (from photographic images) and microscopic VAS score of the reticular dermis.

The inhibition of scarring that may be achieved utilising therapeutically effective amounts of WNT5A, or its therapeutically effective fragments or derivatives, may be of benefit in almost all circumstances where unwanted scarring would otherwise occur.

The following paragraphs are in no way intended to limit the uses to which methods and medicaments of the invention may be put, but may provide useful guidance as to contexts in which it may be wished to inhibit scar formation by use of therapeutically effective amounts of WNT5A or its therapeutically active fragments or derivatives.

The use of methods and medicaments of the invention to inhibit scarring may bring about a notable improvement in the cosmetic appearance of an injured area thus treated. Cosmetic considerations are important in a number of clinical contexts, particularly when wounds are formed at prominent body sites such as the face, neck and hands. Consequently it is a further preferred embodiment that the medicaments and methods of the invention be used to inhibit scarring at sites where it is desired to improve the cosmetic appearance of a scar formed. Indeed, it is a preferred embodiment that the medicaments and methods of the invention be used to inhibit scarring associated with cosmetic surgery. Since the great majority of cosmetic surgeries consist of elective surgical procedures it is readily possible to administer a therapeutically effective amount of WNT5A, or its therapeutically effective fragments or derivatives, prior to surgery, and/or immediately following closure of the wound e.g. with sutures, and this use represents a particularly preferred embodiment of the invention. In the case of elective surgical procedures a preferred route by which WNT5A, or therapeutically effective fragments or derivatives thereof, may be administered is via intradermal injection. Such injections may form raised blebs, which may then be incised as part of the surgical procedure, or alternatively the bleb may be raised by injecting the wound margins after the wound has been closed e.g. by sutures.

The cosmetic outcome of surgical procedures is also an important consideration in plastic surgery, and the use of methods or medicaments of the invention to inhibit scarring associated with plastic surgery constitutes a further preferred embodiment of the invention.

In addition to its cosmetic impact skin scarring is responsible for a number of deleterious effects afflicting those suffering from such scarring. For example, skin scarring may be associated with reduction of physical and mechanical function, particularly in the case of contractile scars (such as hypertrophic scars) and/or situations in which scars are formed across joints. In these cases the altered mechanical properties of scarred skin, as opposed to unscarred skin, and the effects of scar contraction may lead to dramatically restricted movement of a joint (articulation) so effected. Accordingly, it is a preferred embodiment that suitable medicaments and methods of the invention be used to inhibit scarring of wounds covering joints of the body. In another preferred embodiment suitable medicaments and methods of the invention may be used to inhibit scarring of wounds at increased risk of forming a contractile scar.

It is recognised that wounds resulting from burns injuries (which for the purposes of the present invention may be taken to encompass scalding injuries involving hot liquids or gasses) may extend over great areas of an individual so afflicted. Accordingly, burns may give rise to scar formation covering a large proportion of a patient's body. This great extent of coverage increases the risk that the scar formed will cover areas of elevated cosmetic importance (such as the face, neck, arms or hands) or of mechanical importance (particularly the regions covering or surrounding joints). Burns injuries caused by hot liquids are frequently suffered by children (for example as a result of upsetting pans, kettles or the like) and, due to the relatively smaller body size of children, are particularly likely to cause extensive damage over a high proportion of the body area. Furthermore, burns injuries, and particularly those suffered by children, have an elevated risk of producing pathological hypertrophic scars of the type described below. Such hypertrophic scars may increase both the cosmetic and mechanical ill-effects associated with scarring after burns.

It is a preferred embodiment that medicaments and methods of the invention be used to inhibit scarring resulting from burns injuries.

The extent of scar formation, and hence extent of cosmetic or other impairment that may be caused by the scar, may also be influenced by factors such as the tension of the site at which the wound is formed. For example, it is known that skin under relatively high tension (such as that extending over the chest, or associated with lines of tension) may be prone to formation of more severe scars than at other body sites. Thus in a preferred embodiment suitable medicaments and methods of the invention may be used to inhibit scarring of wounds located at sites of high skin tension. There are many surgical procedures that may be used in scar revision to allow realignment of wounds and scars such that they are subject to reduced tension. Probably the best known of these is “Z-plasty” in which two V-shaped flaps of skin are transposed to allow rotation of a line of tension. In a more preferred embodiment such medicaments and methods of the invention be used to inhibit scarring of wounds during surgical revision of disfiguring scars.

Pathological scarring may have more pronounced deleterious effects than arise even as a result of relatively severe normal scarring. Common examples of pathological scars include keloids, hypertrophic scars and pterygium.

Keloid scars (or keloids) constitute a notable example of pathological scarring, and are raised scars that spread beyond the margins of the original wound and invade the surrounding normal skin. Keloids continue to grow over time, do not regress spontaneously, and frequently recur following surgical excision. Keloid scars occur with equal frequency in men and women, mainly from ages 10 to 30, and can result from piercing, surgery, vaccination, tattoos, bites, blunt trauma and burns. A number of studies have suggested that there is an underlying genetic predisposition to keloid formation since keloid scars are more prevalent in dark skinned races.

Keloids appear as elevated scars that may typically be hyperpigmented or hypopigmented in relation to the surrounding tissue. Keloids may be characterised on the basis of their tendency to grow beyond the initial boundaries of a wound. At a microscopic level, keloids may be characterised by the presence of large whorls of collagen, and the predominantly acellular nature of the interior of the lesion.

Hypertrophic scars are raised scars which may have an appearance very similar to keloid lesions. Unlike keloids, hypertrophic scars do not expand beyond the boundaries of the original injury and are not prone to recurrence after excision. Hypertrophic scars may frequently undergo contraction, and it is believed that the contractile nature of hypertrophic scars may be associated with the elevated numbers of myofibroblasts that are frequently reported within these types of scars. Hypertrophic scars may commonly arise as a result of burn or scald injuries, and are particularly common amongst children.

Pterygium is a hypertrophied outgrowth of the subconjunctival tissue to the border of the cornea or beyond. The outgrowth is typically triangular in shape, with the apex pointing towards the pupil. Pterygium may interfere with vision, and may require surgery to remove the hypertrophied tissue. Furthermore, the tissue may frequently re-grow after excision, in the same manner as keloid scars, thus requiring multiple rounds of surgery.

It is recognised that certain types of wound, or certain individuals may be predisposed to pathological scar formation. For instance individuals of Afro-Caribbean, Japanese or Mongloid heritage, or those having a familial history of pathological scarring may be considered to be at increased risk of hypertrophic scar or keloid formation. Wounds of children, and particularly burns wounds of children, are also associated with increased hypertrophic scar formation. Incidences of pterygium may be increased amongst those in whom the eye is frequently exposed to intense sunlight or dust. Accordingly it is a preferred embodiment of the invention that suitable medicaments and methods be used to inhibit scarring of wounds in which there is an increased risk of pathological scar formation.

Although individuals already subject to pathological scarring suffer from a predisposition to further excessive scar formation it is often clinically necessary to surgically revise hypertrophic scars or keloids, with an attendant risk of consequential pathological scar formation. Thus, it is a further preferred embodiment of the invention that the medicaments or methods herein described be used to inhibit scarring of wounds produced by surgical revision of pathological scars.

The ability of WNT5A, and its therapeutically effective fragments and derivatives, to inhibit scarring is of great utility in the treatment of wounds associated with grafting procedures. Treatment using such methods and medicaments of the invention is of benefit both at a graft donor sites and graft recipient sites. The anti-scarring effects of the treatment are able to inhibit scarring that may otherwise occur at sites where tissue for grafting is removed, or that may be associated with the healing and integration of grafted tissue. The inventors believe that the methods and medicaments of the invention confer advantages in the contexts of grafts utilising skin, artificial skin, or skin substitutes.

The majority of the uses considered above relate to the prevention of scarring occurring as a result of penetrative injuries. However, damaging fibrosis may also occur in many tissues as a result of other injuries. In particular, the healing response to crush injuries inflicted on tendons or ligaments may give rise to fibrosis similar to scarring, and the invention is to be taken to encompass the prevention, reduction or inhibition of such fibrosis using the methods or medicaments of the invention.

The healing of wounds involving the peritoneum (the epithelial covering of the internal organs, and/or the interior of the body cavity) may frequently give rise to adhesions. Such adhesions are a common sequitur of surgery involving gynaecological or intestinal tissues, and incidences of adhesion formation may be increased in wounds that are subject to infection (such as bacterial infection) or exposure to radiation. The inventors believe that the ability of the methods and medicaments of the invention to inhibit scarring associated with the healing of such wounds may reduce the occurrence of adhesions. Accordingly, the use of methods and medicaments of the invention to prevent the formation of intestinal or gynaecological adhesions represents a preferred embodiment of the invention. The methods and medicaments of the invention may also be useful in the inhibition of scarring, including formation of adhesions, that may occur on healing of infected wounds or wounds exposed to radiation. Indeed, the skilled person will appreciate that the use of methods or medicaments of the invention in the healing of any wounds involving the peritoneum is a preferred embodiment. Medicaments for this purpose may be administered by lavage, or in a parenteral gel/instillate or locally e.g. from films or carriers inserted at the time of surgery.

Scarring in the eye may be inhibited by medicaments of the invention. In the case of corneal scarring e.g. fully penetrative injuries, LASIK or PRK, application of the medicament may be local e.g. local eye drops, sponge etc. Scarring may additionally be assessed by measuring the opacity, or transmitting/refractory properties, of the cornea e.g. using in vivo confocal microscopy. Scarring deeper in the eye e.g. at the retina (such as at sites of pressure relieving blebs formed in glaucoma surgery) may also be inhibited by the medicaments of the present invention delivered either locally or in devices or by injection.

Scarring in the nervous tissue (either peripheral or central) may be inhibited by the medicaments of the invention. Such scarring may arise as a result of surgery or trauma and may additionally be assessed by future assays of nerve function e.g. sensory or motor tests. Inhibitors of scarring should improve such future outcomes.

Scarring in the blood vessels e.g. following anastomotic surgery, can lead to myointimal hyperplasia and reduction in the volume of the blood vessel lumen. This can be measured directly e.g. using ultrasound, or indirectly be means of blood flow. Inhibition of scarring brought about by medicaments of the invention would lead to a reduction in blood vessel lumen narrowing (restenosis) and a more normal blood flow. Agents of the invention may be delivered to blood vessels by direct injection into the walls of the blood vessel before suturing, by bathing the anastomotic site in the agent or by local devices e.g. stents.

Scarring in tendons and ligaments e.g. following surgery or trauma may be inhibited by medicaments of the invention. Inhibition of scarring may be measured in these terms by restoration of function e.g. ability to bear weight, stretch, flex etc.

Preferred routes of administration by which therapeutically effective amounts of WNT5A, or fragments or derivatives thereof, are administered are discussed more fully elsewhere in the specification, but it may generally be preferred that these therapeutic molecules are provided by local administration to the wound the scarring of which is to be inhibited. Suitable methods by which such local administration may be achieved will depend on the identity of the tissue in question. Preferred routes of administration may include local injection (for example intradermal injection in the case where it is wished to inhibit dermal scarring). Other suitable means of administration include the use of topical medicaments such as sprays; powders; drops (e.g. for the ear or eye); ointments or creams; or release from local devices e.g. stents, implants, polymers.

Medicaments of the invention for the prevention and/or treatment of fibrotic disorders may be formulated and manufactured in any form that allows for the composition to be administered to a patient. Fibrotic disorders will frequently occur in relatively inaccessible tissues and organs, and it may be preferred that WNT5A, or its therapeutically effective fragments or derivatives, be administered systemically. Suitable routes of administration include, without limitation, oral, transdermal, inhalation, parenteral, sublingual, rectal, vaginal and intranasal. By way of example, solid oral formulations (such as tablets or capsules) may be used for the prevention and/or treatment of renal fibrosis or cirrhosis of the liver. Aerosol formulations for inhalation may be preferred for the prevention and/or treatment of chronic obstructive pulmonary disease or other fibrotic disorders of the lungs and airways.

It will be appreciated that many of the routes of administration described above may also be suitable for topical administration to a tissue in which it is wished to prevent and/or treat a fibrotic disorder (for example, inhalation or intranasal for prevention and/or treatment of fibrosis associated with the respiratory system).

Compositions that will be administered to the patient may preferably take the form of one of more dosage units providing a therapeutically effective amount, or a known fraction thereof. Methods of preparing such dosage forms will be well known to the skilled person; for example see Remington's Pharmaceutical Sciences 18^{th} Ed. (1990).

A therapeutically effective amount of WNT5A, or its fragments or derivatives thereof, suitable for use in the methods or medicaments of the invention to prevent and/or treat a fibrotic disorder is an amount that is capable of reducing fibrosis (as assessed with reference to at least at least one of the parameters discussed below) associated with the fibrotic disorder.

A therapeutically effective amount of WNT5A (or a fragment or derivative thereof) may be an amount that is effective to achieve a reduction of at least 10% compared to the level of fibrosis occurring in an untreated tissue subject to the fibrotic disorder. Preferably a therapeutically effective amount may be capable of achieving at least a 20% reduction in fibrosis, more preferably at least 50%, even more preferably at least 75% and most preferably at least a 90% reduction in fibrosis compared to a non-treated fibrotic tissue.

Suitable parameters that may be assessed in the quantification of fibrosis associated with fibrotic disorders will be apparent to the skilled person. The following examples are provided by way of illustration only.

Fibrosis associated with fibrotic disorders may classically be assessed with reference to trichrome staining (for example Masson's trichrome or Mallory's trichrome) of biopsy samples taken from a tissue subject to the fibrotic disorder. These samples may be compared with non-fibrotic tissues and reference tissues representative of staining in a range of tissues subject to fibrosis. Protocols for trichrome staining are well known to the skilled person, and kits that may be used to conduct trichrome staining are commercially available.

It will be appreciated that in many cases it may be preferred to avoid invasive procedures such as the collection of biopsies. In recognition of this fact a number of non-invasive procedures have been devised that allow assessment of fibrosis withouth the need for biopsy samples. Examples of such procedures include Fibrotest (FT) and Actitest (AT).

These commercially available assays use five or six biochemical markers of fibrosis for use as a non-invasive alternative to liver biopsy in patients with chronic hepatitis C or B, alcoholic liver disease and metabolic steatosis (for instance the overweight, patients with diabetes or hyperlipidemia). Through use of such biochemical markers, and analysis using selected algorithms, these procedures are able to determine levels of liver fibrosis and necroinflammatory activity. The use of such tests is increasingly clinically accepted as an alternative to biopsies, and the tests are commercially available from suppliers such as BioPredictive.

The methods or medicaments of the invention may be used prophylactically, i.e. prior to wounding that would otherwise lead to scar formation, or prior to the onset of a fibrotic disorder.

In the case of the inhibition of scarring, this may involve administration of a therapeutically effective amount of WNT5A, or a therapeutically effective fragment or derivative thereof, at sites where no wound presently exists, but where a wound that would otherwise give rise to a scar is to be formed. By way of example medicaments in accordance with the invention may be administered to sites that are to undergo wounding as a result of elective procedures (such as surgery), or to sites that are believed to be at elevated risk of wounding.

It may be preferred that the medicaments of the invention are administered to the site, around the time of wounding, or immediately prior to the forming of a wound (for example in the period up to six hours before wounding) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may determined with reference to factors such as the patient's age, health, and predisposition to healing complications or adverse scarring). The prophylactic use of methods and medicaments in accordance with the invention is a preferred embodiment of the invention, and is particularly preferred in the prevention, reduction or inhibition of scarring in the context of surgical wounds.

In the case of the treatment of fibrotic disorders medicaments of the invention may be administered to a site at elevated risk of developing a fibrotic disorder prior to formation of said disorder. Suitable sites may be those that are perceived to be at elevated risk of the development of fibrotic disorders. An elevated risk of development of fibrotic disorders may arise as a result of disease, or as a result of environmental factors (including exposure to fibrotic agents), or as a result of genetic predisposition.

In the same way as for the inhibition of scarring discussed above, medicaments of the invention may be administered immediately prior to onset of a fibrotic disorder, or at an earlier time. The skilled person will be able to establish the optimal time for administration of medicaments of the invention used to treat fibrotic disorders using standard techniques, in the same manner as for inhibition of scarring.

The methods and medicaments of the invention are also able to inhibit scarring if administered after a wound has already been formed. It is preferred that such administration should occur as early as possible after formation of the wound, but agents of the invention are able to inhibit scarring at any time up until the healing process has been completed (i.e. even in the event that a wound has already partially healed the methods and medicaments of the invention may be used to inhibit scarring in respect of the remaining un-healed portion). It will be appreciated that the “window” in which the methods and medicaments of the invention may be used to inhibit scarring is dependent on the nature of the wound in question (including the degree of damage that has occurred, and the size of the wounded area). Thus, in the case of a large wound, the methods and medicaments of the invention may be administered relatively late in the healing response yet still be able to inhibit scarring, as a consequence of the relatively prolonged time that large wounds require to heal.

The methods and medicaments of the invention may, for instance, preferably be administered within the first 24 hours after a wound is formed, but may still inhibit scarring if administered up to ten, or more, days after wounding.

Similarly, the methods and medicaments of the invention may be administered to a site at which a fibrotic disorder is already developing, in order to prevent further fibrosis taking place. This use will obviously be advantageous in situations in which the degree of fibrosis that has occurred prior to administration of WNT5A, or a therapeutically effective fragment or derivative thereof, is sufficiently low that the fibrotic tissue is still able to function.

Medicaments of the invention may preferably be administered within 24 hours of the onset of fibrosis, but may still be effective if administered considerably later in the fibrotic process. For example, medicaments may be administered within a month of the onset of fibrosis (or of the diagnosis that fibrosis is taking place), or within sixth months, or even one or more years, depending on the extent of fibrosis that has already occurred, the proportion of the tissue effected by the fibrotic disorder, and the rate at which the fibrotic disorder is progressing.

The methods and medicaments of the invention may be administered on one or more occasions (as necessary) in order to inhibit scarring, and/or prevent and/or treat a fibrotic disorder.

For instance, in the case of inhibition of scarring, therapeutically effective amounts of the medicaments may be administered to a wound as often as required until the healing process has been completed. By way of example, the medicaments of the invention may be administered daily or twice daily to a wound for at least the first three days following the formation of the wound. The inventors have found that regimes involving two administrations of medicaments of the invention, the first prior to formation of a wound and the second after wounding, are particularly beneficial in inhibiting scar formation. Preferably such regimes may involve a first administration immediately prior to formation of a wound and a second administration 24 hours after wounding.

Most preferably the methods or medicaments of the invention may be administered both before and after formation of a wound. The inventors have found that administration of the medicaments of the invention immediately prior to the formation of a wound, followed by daily administration of such agents for one or more days following wounding, is particularly effective in inhibiting scarring and/or treating fibrotic disorders.

In the case where WNT5A, or its therapeutically effective fragments or derivatives, are used to prevent and/or treat fibrotic disorders, a therapeutically effective amount sufficient to prevent the onset or progression of a fibrotic disorder may be provided by means of a number of administrations. Suitable regimes may involve administration monthly, weekly, daily or twice daily.

The inventors believe that WNT5A, or therapeutically effective fragments or derivatives thereof, may also be used to reduce existing scars. Accordingly the use of methods and medicaments of the invention in the reduction of existing scars constitutes a preferred use according to the invention. A therapeutically effective amount of WNT5A, its fragments or derivatives, may be provided by means of any number of suitable administrations. Suitable regimes for these administrations may be readily devised by the skilled person using techniques (including in vitro studies, animal and human studies) well known in and established within the pharmaceutical industry.

WNT5A, or its therapeutically effective fragments or derivatives, may also be used in the treatment of existing fibrotic disorders in order to bring about a reduction in the extent of fibrosis associated with the disorder. Therapeutically effective amounts, and preferred administration regimes, for use in this manner may be determined as described elsewhere in the specification.

For the purposes of the present specification by “agent” or “agent of the invention” is meant WNT5A, or a therapeutically effective fragment of WNT5A, or a therapeutically effective derivative of WNT5A. It will be appreciated that all such agents may be incorporated in medicaments in accordance with the invention, and all may be used in the methods or uses of the invention. The medicaments of the invention represent preferred compositions by which WNT5A, or its therapeutically effective fragments or derivatives, may be administered in order to put the methods of the invention into practice.

It will be appreciated that the amount of a medicament of the invention that should be provided to a wound or fibrotic disorder, in order that a therapeutically effective amount of WNT5A, its fragments or derivatives, may be administered, depends on a number of factors. These include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the agent in the subject being treated.
B) The specific condition to be treated (e.g. acute wounding or chronic fibrotic disorders).
C) The age of the subject.
D) The size of the site to be treated.

The frequency of administration will also be influenced by the above-mentioned factors and particularly the half-life of the chosen agent within the subject being treated.

Generally when medicaments in accordance with the invention are used to treat existing wounds or fibrotic disorders the medicament should be administered as soon as the wound occurs or the fibrotic disorder begins. In the case of wounds or fibrotic disorders that are not immediately apparent, such as those at internal body sites, medicaments may be administered as soon as the wound or disorder is diagnosed. Therapy with methods or medicaments in accordance with the invention should continue until scarring has been inhibited, or the fibrotic disorder prevented or treated, to a clinician's satisfaction.

Frequency of administration will depend upon the biological half-life of the agent used. Typically a cream or ointment containing an agent of the invention should be administered to a target tissue such that the concentration of the agent at a wound or site of fibrosis is maintained at a level suitable for having a therapeutic effect. This may require administration daily or even several times daily. The inventors have found that administration of an agent of the invention immediately prior to wounding, with a further administration one day after wounding is particularly effective for the inhibition of scarring.

Medicaments of the invention, may be administered by any suitable route capable of achieving the desired effect of inhibiting scarring or treating fibrosis, but it is preferred that the medicaments be administered locally at a wound site or site of a fibrotic disorder.

The inventors have found that the inhibition of scarring may be effected by the administration of an agent of the invention by injection at a wound site or site of a fibrotic disorder. For instance, in the case of dermal wounds or dermal fibrosis, agents of the invention may be administered by means of intradermal injection. Thus a preferred medicament in accordance with the invention comprises an injectable solution of an agent of the invention (e.g. for injection around the margins of a wound, or a site likely to be wounded). Suitable formulations for use in this embodiment of the invention are considered below.

Alternatively, or additionally, medicaments of the invention may also be administered in a topical form to inhibit scarring or treat a fibrotic disorder. In the case of inhibiting scarring, such administration may be effected as part of the initial and/or follow up care for the wounded area.

The inventors have found that inhibition of scarring or treatment of fibrosis can be very beneficially effected by topical application of an agent of the invention to a wound or fibrotic disorder (or, in the case of prophylactic application, to a tissue or site where a wound or fibrotic disorder will occur).

Compositions or medicaments containing agents of the invention may take a number of different forms depending, in particular, on the manner in which they are to be used. Thus, for example, they may be in the form of a liquid, ointment, cream, gel, hydrogel, powder or aerosol. All of such compositions are suitable for topical application to a wound or fibrotic disorder, which is a preferred means of administering agents of the invention to a subject (person or animal) in need of treatment.

The agents of the invention may be provided on a sterile dressing or patch, which may be used to cover a wound or fibrotic site to be treated.

The agents of the invention may be released from a device or implant, or may be used to coat such a device e.g. stent.

It will be appreciated that the vehicle of a composition comprising agents of the invention should be one that is well tolerated by the patient and allows release of the agent to the wound or fibrotic site. Such a vehicle is preferably biodegradeable, bioresolveable, bioresorbable and/or non-inflammatory.

Medicaments and compositions comprising agents of the invention may be used in a number of ways. Thus, for example, a composition may be applied in and/or around a wound or fibrotic disorder in order to inhibit scarring or treat fibrosis. If the composition is to be applied to an existing wound or fibrotic site, then the pharmaceutically acceptable vehicle will be one which is relatively “mild” i.e. a vehicle which is biocompatible, biodegradable, bioresolvable and non-inflammatory.

An agent of the invention, or a nucleic acid encoding such an agent (as considered further below), may be incorporated within a slow or delayed release device. Such devices may, for example, be placed on or inserted under the skin and the agent or nucleic acid may be released over days, weeks or even months.

Delayed release devices may be particularly useful for patients, such as those suffering from extensive or pathological scarring or from long-lasting fibrotic disorders, who require long-term administration of therapeutically effective amounts of WNT5A, its fragments or derivatives. Such devices may be particularly advantageous when used for the administration of an agent or nucleic acid that would otherwise normally require frequent administration (e.g. at least daily administration by other routes).

Daily doses of an agent of the invention may be given as a single administration (e.g. a daily application of a topical formulation or a daily injection). Alternatively, the agent of the invention may require administration twice or more times during a day. In a further alternative, a slow release device may be used to provide optimal doses of an agent of the invention to a patient without the need to administer repeated doses.

A dose of a composition comprising agents of the invention may preferably be sufficient to provide a therapeutically effective amount of WNT5A, its fragments or derivatives, in a single administration. However, it will be appreciated that each dose need not in itself provide a therapeutically effective amount of an agent of the invention, but that a therapeutically effective amount may instead be built up through repeated administration of suitable doses.

Various suitable forms are known for compositions comprising agents of the invention. In one embodiment a pharmaceutical vehicle for administration of an agent of the invention may be a liquid and a suitable pharmaceutical composition would be in the form of a solution. In another embodiment, the pharmaceutically acceptable vehicle is a solid and a suitable composition is in the form of a powder or tablet. In a further embodiment the agent of the invention may be formulated as a part of a pharmaceutically acceptable transdermal patch.

A solid vehicle can include one or more substances that may also act as flavouring agents, lubricants, solubilizers, suspending agents, fillers, glidants, compression aids, binders or tablet-disintegrating agents; it can also be an encapsulating material. In powders, the vehicle is a finely divided solid that is in admixture with the finely divided agent of the invention. In tablets, the agent of the invention is mixed with a vehicle having the necessary compression properties in suitable proportions and compacted in the shape and size desired. The powders and tablets preferably contain up to 99% of the agent of the invention. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions. The agent of the invention can be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intrathecal, epidural, intraperitoneal, intradermal, intrastromal (cornea), intraadventitial (blood vessels) or subcutaneous injection. Sterile solutions can also be administered intravenously. The agent of the invention may be prepared as a sterile solid composition that may be dissolved or suspended at the time of administration using sterile water, saline, or other appropriate sterile injectable medium. Vehicles are intended to include necessary and inert binders, suspending agents, lubricants and preservatives.

In the situation in which it is desired to administer an agent of the invention by means of oral ingestion, it will be appreciated that the chosen agent will preferably be an agent having an elevated degree of resistance to degradation. For example, the agent of the invention may be protected (for instance using the techniques described above) so that its rate of degradation in the digestive tract is reduced.

Compositions of agents of the invention are suitable to be used for inhibiting scarring or treating fibrosis in the cornea. Corneal wounds may result from trauma to the eye arising as a result of accidental injury or as a result of surgical operations (e.g. laser surgery on the cornea). In the case of administration of agents of the invention to the outer surfaces of the eye, such as the cornea, a preferred medicament of the invention may be in the form of an eye drop.

Fibrosis in the eye may be associated with conditions such as proliferative vitreoretinopathy. In the event that it is wished to prevent fibrosis associated with vitreoretinopathy, such as proliferative vitreoretinopathy, it may be preferred to administer WNT5A, or a therapeutically effective fragment or derivative thereof, by means of intravitreal injection or localised (e.g. intraocular) release device. Such injections may preferably follow surgery or intravitreal implantation procedures.

Agents of the invention may be used in a range of “internal” wounds or fibrotic disorders (i.e. wounds or fibrotic disorders occurring within the body, rather than on an external surface). Examples of internal wounds include penetrative wounds that pass through the skin into underlying tissues, and wounds associated with surgical procedures conducted within the body. The range of fibrotic disorders that effect internal sites is extensive, and includes lung fibrosis, liver fibrosis, kidney fibrosis and muscle fibrosis.

In a preferred example, medicaments in accordance with the invention for use in the treatment of wounds or fibrotic disorders arising in the lungs or other respiratory tissues may be formulated for inhalation.

In a preferred example, medicaments in accordance with the invention for use in the treatment of wounds or fibrotic disorders arsing in the body cavities e.g. abdomen, pelvis may be formulated as a lavage, gel or instillate.

Known procedures, such as those conventionally employed by the pharmaceutical industry (e.g. in vivo experimentation, clinical trials etc), may be used to establish specific formulations of compositions comprising agents of the invention and precise therapeutic regimes for administration of such compositions (such as daily doses of the active agent and the frequency of administration).

A suitable dose of an agent in accordance with the invention able to inhibit scarring or treat a fibrotic disorder may depend upon a range of factors including (but not limited to) the nature of the tissue to be treated, the area and/or depth of the wound or fibrosis to be treated, the severity of the wound or fibrosis, and the presence or absence of factors predisposing to pathological scar formation.

The inventors believe that the amount of WNT5A, or a therapeutically active fragment or derivative thereof, that may be administered to a wound or site of fibrosis in a single incidence of treatment (for instance by topical administration such as intradermal injection) may preferably be above about 2 fmoles/cm of wound or cm² fibrosis, and below about 22 pmoles/cm of wound or cm² fibrosis.

By way of further example, the preferred amount of WNT5A, or a therapeutically active fragment or derivative thereof, to be administered to a wound or site of fibrosis over a period of approximately 24 hours (for instance by topical administration such as intradermal injection) may preferably be above about 4 (moles/cm of wound or cm² fibrosis, and below about 44 pmoles/cm of wound or cm² fibrosis.

The skilled person will appreciate that the suggestions above are provided for guidance. In particular it will be appreciated that the amount of WNT5A, or a therapeutically effective fragment or derivative thereof, to be administered via topical administration may be altered depending on permeability of the tissue or organ to which the topical composition is administered. Thus in the case of relatively impermeable tissues or organs it may be preferred to increase the amount of WNT5A, or therapeutically effective fragment or derivative thereof, administered. Such an increased amount of WNT5A, or a therapeutically effective fragment or derivative thereof, may still represent a therapeutically effective amount, if the amount of the agent taken up into the tissue or organ to be treated is therapeutically effective (i.e. if a therapeutically effective amount permeates the tissue or organ to be treated, irrespective of the fact that a larger, non-therapeutic, amount of the agent may remain on the surface of, and unable to penetrate, the tissue or organ being treated).

The inventors believe that the amount of WNT5A, or a therapeutically active fragment or derivative thereof, to be administered to a wound or site of fibrosis in a single incidence of treatment will preferably not exceed 21 pmoles/cm of wound, or cm² of fibrosis. More preferably the amount administered in a single incidence of treatment will be less than 21 pmoles/cm of wound, or cm² of fibrosis.

Preferably, the amount of WNT5A, or a therapeutically active fragment or derivative thereof, to be administered to a wound or site of fibrosis over a period of approximately 24 hours will not exceed 44 pmoles/cm of wound, or cm² of fibrosis. More preferably the amount to be administered over a period of approximately 24 hours will be less than 44 pmoles/cm of wound.

Preferably the total amount of WNT5A, or a therapeutically active fragment or derivative thereof, administered to a wound or site of fibrosis will not exceed 44 pmoles/cm of wound, or cm² of fibrosis.

The total amount of WNT5A, or a therapeutically active fragment or derivative thereof, that may be administered by local injection to a wound or site of fibrosis may be preferably be in the region of 0.01-44 pmoles/centimetre of wound or cm² fibrosis.

Preferably the total amount of WNT5A, or a therapeutically active fragment or derivative thereof, that may be administered by local injection to a wound or site of fibrosis may be preferably be in the region of 0.022-0.44 pmoles/centimetre of wound or cm² fibrosis.

In the case of topical application of WNT5A, or a therapeutically active fragment or derivative thereof, to wounds or sites of fibrosis, a suitable amount to be administered may be in the region of 0.01-44 pmoles/cm of wound or cm² or fibrosis.

Preferably, for topical application of WNT5A, or a therapeutically active fragment or derivative thereof, to wounds or sites of fibrosis, a suitable amount to be administered may be in the region of 0.022-0.44 pmoles/cm of wound or cm² or fibrosis.

WNT5A, or a therapeutically active fragment or derivative thereof, may preferably be provided in the form of an injectable solution at a concentration of between 0.22 nM and 50 nM. Preferably approximately 1004, of such a solution administered per centimetre of wound or cm² of fibrosis over a 24 hour period.

The WNT5A, or therapeutically active fragments or derivatives thereof, may more preferably be administered as a 0.11 nM to 165 nM solution. 100 μL of such a solution may be administered per centimetre of wound or cm² of fibrosis over a 24 hour period.

Most preferably the WNT5A, or therapeutically active fragments or derivatives thereof, may be administered as a 0.11 nM; 2.2 nM; or 165 nM solution with 100 μL of such a solution administered per centimetre of wound or cm² of fibrosis over a 24 hour period.

The skilled person will appreciate that effective therapeutic amounts of WNT5A, or its fragments or derivatives, may be determined with reference to the concentration of the agent that is attained in the organ or tissue to which they are administered. The information regarding therapeutically effective dosages set out herein will provide sufficient guidance to allow the skilled person to calculate the local concentrations of WNT5A, or its therapeutically effective fragments or derivatives, established by intradermal injection, and, based on these values, to determine suitable amounts of such agents that may be administered by other routes in order to achieve equivalent local concentrations.

The inventors have found that WNT5A may be administered by way of an injectable solution containing between 0.5 ng/100 μL and 750 ng/100 μL of an agent of the invention in order to inhibit scarring or treat fibrosis when administered as an intradermal injection providing 104 μL of solution per cm of wound margin or cm² of fibrosis.

It will be appreciated that the guidance as to doses and amounts of WNT5A, or its therapeutically effective fragments or derivatives, provided above is applicable both to medicaments of the invention, and also to the methods of the invention.

In the case where the paragraphs above consider the administration of a specified amount of a medicament per cm of a wound it will be appreciated that this may be administered to either one or both of the margins of a wound to be treated.

The skilled person will recognise that the information provided in the preceding paragraphs as to amounts or volumes of WNT5A, or therapeutically effective fragments or derivatives thereof, which may be administered to wounds or sites of fibrotic disorders, may be varied by the skilled practitioner in response to the specific clinical requirements of an individual patient. For example, it will be appreciated that in the case of particularly deep or wide wounds the amounts provided by way of guidance above may be varied upwards, while still providing a therapeutically effective amount of WNT5A, or fragment or derivative thereof. Suitable variations based on the guidance provided above will be readily apparent to those of skill in the art.

Agents of the invention may be used to inhibit scarring or treat fibrosis as a monotherapy (e.g. through use of medicaments of the invention alone). Alternatively the methods or medicaments of the invention may be used in combination with other compounds or treatments for the inhibition of scarring or treatment of fibrosis. Suitable compounds that may be used as parts of such combination therapies will be well known to those skilled in the art.

The skilled person will appreciate that therapeutically effective amounts of agents such as WNT5A, or its fragments or derivatives, may be administered at the sites of wounds or fibrotic disorders where it is wished to inhibit scarring or treat fibrosis by virtue of cellular expression (commonly referred to as gene therapy). Such cellular expression must be controlled in order to prevent the accumulation of non-therapeutic amounts of such agents, or even amounts that are capable of exacerbating scarring or fibrosis. Accordingly, the invention provides a method of inhibiting scar formation, the method comprising inducing cellular expression of a therapeutically effective amount of WNT5A, or a fragment or derivative thereof, at a site where scarring is to be inhibited. The invention further provides a method of treating a fibrotic disorder, the method comprising inducing cellular expression of a therapeutically effective amount of WNT5A, or a fragment or derivative thereof. In both these aspects the key inventive concept lies in the regulation of cellular expression such that a therapeutically effective amount of WNT5A, or its fragments or derivatives, is obtained.

Based on the teaching contained in the present specification, setting out for the first time the key fact that only certain amounts or concentrations of WNT5A (or its fragments or derivatives) are therapeutically effective, it will be a matter of routine experimentation for one skilled in the art to devise protocols by which cells may be induced to express therapeutically effective amounts of WNT5A (or its fragments or derivatives).

For example, the skilled person will appreciate that such cellular expression of therapeutically effective amounts of WNT5A may be achieved by manipulating naturally occurring expression of this molecule by cells in the region of the wound or site of fibrosis to be treated.

Alternatively, and preferably, cells in the region of wound or site of fibrosis that is to be treated may be induced to express WNT5A, or therapeutically effective fragments or derivatives thereof, by means of the introduction of materials encoding such agents. Suitable materials may typically comprise nucleic acids such as DNA or RNA, and these may be devised based upon the sequences set out in Sequence ID Nos. 1 and 2.

Nucleic acids for use in this embodiment of the invention may be administered “as is”, for example by means of ballistic transfection, or as parts of a larger construct, which may be able to incorporate stably into cells so transfected. Suitable constructs may also contain regulatory elements, by which expression of a therapeutically effective amount of WNT5A, or a fragment or derivative thereof, may be achieved.

The invention will now be further described by way of example with reference to the following experimental protocols and studies, and the accompanying Sequence Information section and Figures in which:

The Sequence Information section shows the amino acid sequence of human WNT5A (shown as Sequence ID NO. 1), and the sequence of cDNA encoding human WNT5A (shown as Sequence ID No. 2).

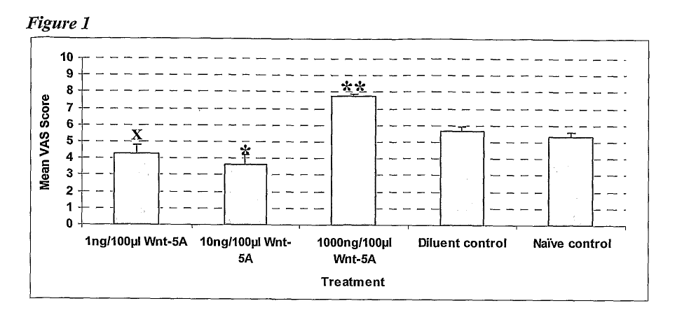

FIG. 1 compares macroscopic VAS scores of treated scars, untreated scars and control treated scars. Results are shown in the form of a bar chart showing macroscopic VAS scores for scars formed 70 days after wounding. “**” indicates p<0.01 versus naïve and diluent controls. “*” indicates p<0.05 versus naïve and diluent control. “^{X}” indicates p<0.05 versus diluent control only (not naïve control)

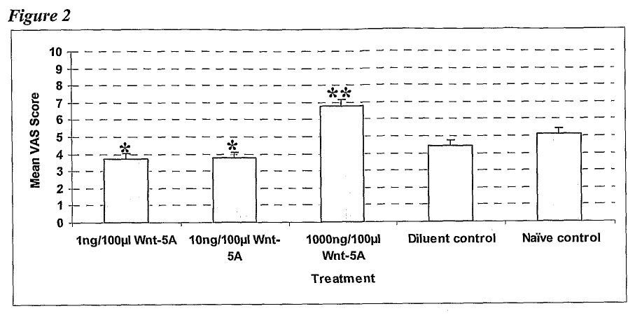

FIG. 2 compares microscopic VAS scores of treated scars, untreated scars and control treated scars. Results are shown in the form of a bar chart showing microscopic VAS scores for scars formed 70 days after wounding. “*” indicates p<0.01 versus naïve and diluent control. “*” indicates p<0.01 versus naïve control only (not diluent control).

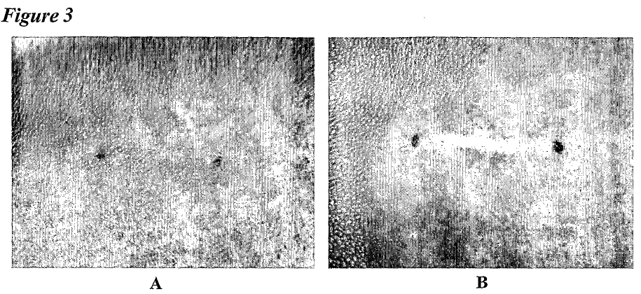

FIG. 3 shows illustrative macroscopic images of a treated scar (Panel A); and an untreated scar (Panel B). The photographs show macroscopic appearance of scars 70 days after incision. The scar in panel A was treated with WNT5A at a concentration of 10 ng/100 μl, while the wound producing the scar shown in panel B was untreated (naïve wound).

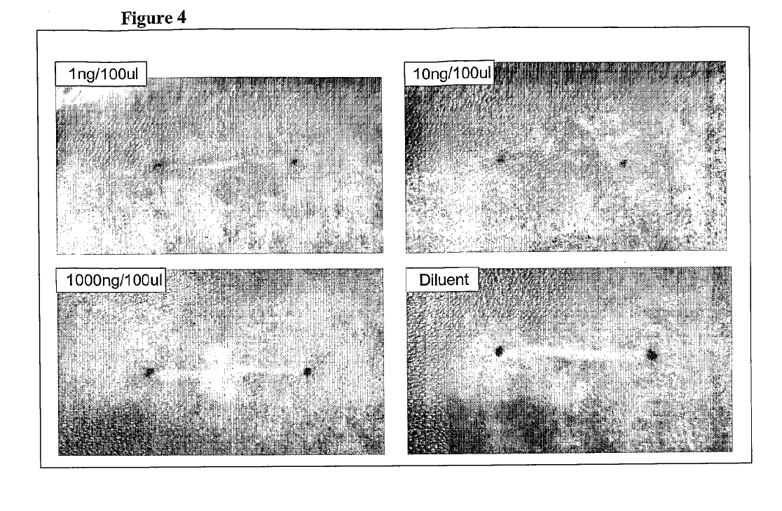

FIG. 4 shows illustrative macroscopic images of treated scars (Panels A, B and C); and a control treated scar (Panel D). The photographs show macroscopic appearance of scars 70 days after incision. The wounds have been treated with 1 ng/100 μl WNT5A, 10 ng/100 μl WNT5A or 1000 ng/100 μl WNT5A, or with diluent alone, as shown in the captions.

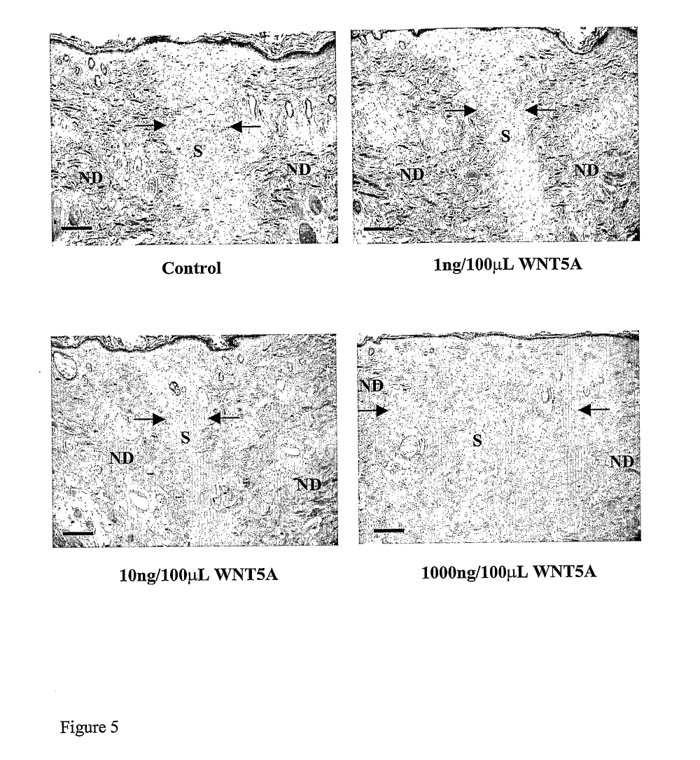

FIG. 5 shows illustrative microscopic images comparing scars produced on healing of control treated wounds and wounds treated with different amounts of WNT5A (including both therapeutically effective amounts and non-therapeutically effective amounts). Images were taken from cutaneous scars of rats 70 days post-wounding and treatment. The images were taken, at ×5 magnification, from histological sections of scars stained with Masson's Trichrome. Arrows indicate the edges of resultant scars following full thickness incisional wounding and treatment. ‘S’ indicates the scar, ‘ND’ indicates the normal surrounding dermis.

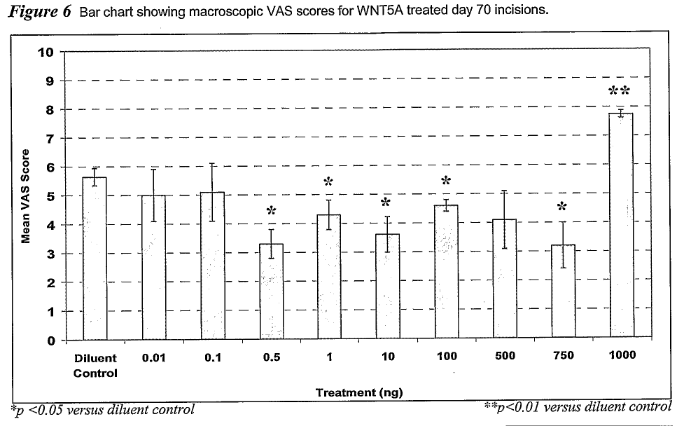

FIG. 6 compares macroscopic VAS scores of treated scars and control treated scars. Results are shown in the form of a bar chart showing macroscopic VAS scores for scars formed 70 days after wounding. “**” indicates p<0.01 versus diluent controls. “*” indicates p<0.05 versus diluent control.

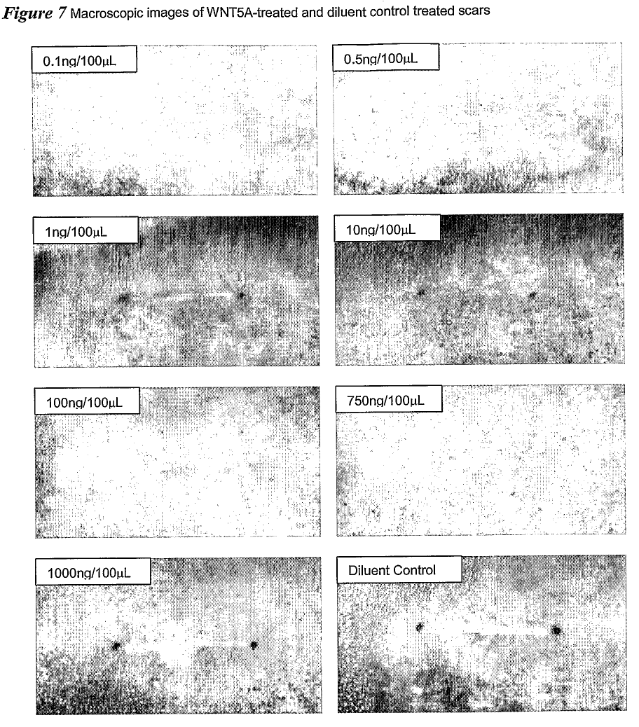

FIG. 7 shows representative macroscopic images illustrating scars produced on healing of control treated wounds and wounds treated with WNT5A (as shown in the captions). The scars were photographed 70 days after experimental incisions had been formed.

### EXPERIMENTAL RESULTS 1

The inventors investigated the effects of WNT5A on scarring using in an in vivo model of scar formation and healing.

### 1.1 Incisional Wound Healing Model and Treatment with WNT5A

Recombinant mouse WNT5A (Catalogue number #645-WN/CF; Lot MCR074091) was purchased from R&D Systems.

The WNT5A was diluted in phosphate buffered saline (PBS) to produce three solutions having concentrations as follows:
1. 1 ng/100 μl (a 0.22 nM solution);
2. 10 ng/100 μl (a 2.2 nM solution); and
3. 1000 ng/100 μl (a 220 nM solution).

PBS alone was used as a diluent control.

Adult male Sprague Dawley rats weighing 225-250 g were anaesthetised using halothane, oxygen and nitrous oxide. At day 0, wound sites were marked on the dorsal skin and wound sites were injected intradermally with 100 μl of the WNT5A or PBS solutions prior to wounding. The intradermal injections caused the formation of a raised bleb, which was then incised to form 1 cm long experimental wounds. Naïve wounds (also 1 cm in length) received no treatment.

Treated and control-treated wounds were re-treated day 1 post-wounding with the appropriate treatment via injection of 50 μl to each of the two margins of the wound.

Accordingly, each injection of the 1 ng/100 μl solution provided 0.022 pmoles of WNT5A, whilst each injection of the 10 ng/100 μl solution provided 0.22 pmoles of WNT5A, and each injection of the 1000 ng/100 μl solution provided 22 pmoles of WNT5A.

Diluent control treated wounds received PBS alone (administered in the same volume and by the same route as WNT5A solutions in treated wounds), and the naïve control wounds received no treatment.

### 1.2 Assessment of Scarring

70 days after wounding the experimental rats were killed, and the scars resulting from treated wounds and control wounds assessed both macroscopically and microscopically.

The scars of the experimental rats were photographed and assessed using macroscopic scar assessment sheets. Macroscopic assessment of scarring was carried out using a visual analogue scale (VAS) consisting of a 0-10 cm line representing a scale, from left to right, of 0 (corresponding to normal skin) to 10 (indicative of a bad scar). A mark was made by an assessor on the 10 cm line based on an overall assessment of the scar taking into account parameters such as the height, width, contour and colour of the scar. The best scars (typically of small width with colour, height and contour like normal skin) were scored towards the normal skin end of the scale (the left hand side of the VAS line) and bad scars (typically large width, raised with uneven contours and whiter colour) were scored towards the bad scar end of the scale (the right hand side of the VAS line). The marks were measured from the left hand side to provide the final value for the scar assessment in centimetres (to 1 decimal place).

For microscopic assessment the scars were excised from the experimental rats (incorporating a small amount of surrounding normal tissue) and fixation of the scars in 10% (v/v) buffered formal saline. The fixed tissue was then processed for wax histology. Histological slides were stained using Masson's trichrome, and scarring assessed using a microscopic visual analogue scale (VAS). This consisted of a 0-10 cm line representing a scale, from left to right, of 0 (corresponding to normal skin) to 10 (indicative of a bad scar). A mark was made on the 10 cm line based on an overall assessment of the scar taking into account parameters such as collagen fibre spacing, orientation and thickness. The best scars (typically narrow scars with thick and randomly organised collagen fibres that have normal spacing between fibres, similar to the surrounding normal dermis) were scored towards the normal skin end of the scale (the left hand side of the VAS line) and bad scars (typically wide scars with thin densely packed parallel collagen fibres) were scored towards the bad scar end of the scale (the right hand side of the VAS line). The marks were measured from the left hand side to provide the final value for the scar assessment in centimetres (to 1 decimal place).

A comparison of the macroscopic VAS scores of scars formed on healing of WNT5A treated wounds and control treated wounds is shown in FIG. 2.

A comparison of the microscopic VAS scores of scars formed on healing of WNT5A treated wounds and control treated wounds is shown in FIG. 3.

Representative images showing the macroscopic appearance of scars formed on healing of WNT5A treated wounds and control treated wounds are shown in FIGS. 4 and 5.

Representative images showing the macroscopic appearance of scars formed on healing of WNT5A treated wounds and control treated wounds are shown in FIG. 6.

### 1.3 Results

Both macroscopic and microscopic analysis of scars formed from incisional wounds (assessed at 70 days post-wounding) showed that the addition of WNT5A to wounds was able to significantly inhibit scarring. Surprisingly, the results show that when WNT5A is administered in an excessive amount the effect of such administration is to exacerbate scarring, rather than to inhibit scarring.

Administration of WNT5A at concentrations of 1 ng/100 μl and 10 ng/100 μl significantly inhibited scarring, as compared to controls (p<0.05), when assessed using either a macroscopic or microscopic VAS. These results, which are shown in FIGS. 2 and 3, arise as a result of improvements brought about in the abundance and orientation of ECM components in the dermis. These microscopic improvements significantly improve the macroscopic appearance of scars formed.

That scarring is effectively inhibited by these concentrations of WNT5A is clearly illustrated in FIGS. 4, 5 and 6. FIGS. 4 and 5 show representative macroscopic images of treated and control treated scars. Scars resulting from wounds treated with therapeutically effective amounts of WNT5A are considerably more difficult to detect than the scars produced on healing of untreated and control treated wounds. FIG. 6 shows representative microscopic images from histological slides of scars produced on healing of WNT5A treated and untreated wounds. These show that the deposition of extracellular matrix molecules, such as collagen, found in WNT5A treated scars more closely approximates the arrangement in unwounded skin than does the extracellular matrix in untreated scars. This difference (i.e. this increased similarity to unwounded skin) is evident with reference to both the quantity of collagen present and the orientation of said collagen.

In contrast, administration of WNT5A at 1000 ng/100 μl significantly increased scarring when compared to controls (p<0.01). This effect was noted with reference to both microscopic and macroscopic assessment, as shown in FIGS. 2 and 3. The increased scarring produced on administration of WNT5A at 1000 ng/100 μl can be seen both macroscopically and microscopically in FIG. 5 and FIG. 6.

### EXPERIMENTAL RESULTS 2

### 2.1 Target Molecule and Dose Details

Recombinant mouse WNT5A was purchased from R&D Systems (Catalogue number #645-WN/CF; Batch No: MCR136031.

Molecular Mass: 45 kDa by SDS-PAGE (predicted MW is 38 kDa)

| Weight/Volume | Molar Concentration | | Moles | |
|---|---|---|---|---|
| 0.01 ng/100 μL | 2.2 | pM | 0.2 | fMoles |
| 0.1 ng/100 μL | 22 | pM | 2 | fMoles |
| 0.5 ng/100 μL | 0.11 | nM | 0.01 | pMoles |
| 1 ng/100 μL | 0.22 | nM | 0.02 | pMoles |
| 10 ng/100 μL | 2.2 | nM | 0.22 | pMoles |
| 100 ng/100 μL | 22 | nM | 2.2 | pMoles |
| 500 ng/100 μL | 110 | nM | 11 | pMoles |
| 750 ng/100 μL | 165 | nM | 16.5 | pMoles |
| 1000 ng/100 μL | 222.2 | nM | 22.2 | pMoles |

### 2.2 Experimental Results

The WNT5A was diluted in phosphate buffered saline (PBS) to produce nine solutions having concentrations as follows:
1. 0.01 ng/100 μl (a 2.2 pM solution);
2. 0.1 ng/100 μl (a 22 pM solution);
3. 0.5 ng/100 μl (a 0.11 nM solution);
4. 1 ng/100 μl (a 0.22 nM solution);
5. 10 ng/100 μl (a 2.2 nM solution);
6. 100 ng/100 μl (a 22 nM solution);
7. 500 ng/100 μl (a 110 nM solution);
8. 750 ng/100 μl (a 165 nM solution); and
9. 1000 ng/100 μl (a 222 nM solution).

PBS alone was used as a diluent control.

At day 0, adult male Sprague Dawley rats weighing 225-250 g were anaesthetised using halothane, oxygen and nitrous oxide. Prophylactic analgesia was administered as 10 μg/kg of Buprenorphine subcutaneously. The dorsum of the rats were shaved and wound sites marked according to the Renovo rat incisional wounding template (2×1 cm wounds at 5 cm from the base of the skull and 1 cm from the midline in each rat; wound positions A and B). Wound sites were marked on the dorsal skin and wound sites were injected intradermally with 100 μl of the WNT5A (active) or PBS (diluent control) solutions prior to wounding. The intradermal injections caused the formation of a raised bleb, which was then incised to form 1 cm long experimental wounds. Both wounds were dosed again 1 day after surgery, with either WNT5a or PBS (50 μl of treatment/wound margin; total of 100 μl/wound site). Animals were housed singly for 7 days post-surgery and thereafter housed in pairs and given food and water ad libitum until harvest at day 70 post-wounding.

Accordingly, each injection of the 0.01 ng/100 μl solution provided 0.2 fmoles of WNT5A; each injection of the 0.1 ng/100 μl solution provided 2 fmoles of WNT5A; each injection of the 0.5 ng/100 μl solution provided 0.01 pmoles of WNT5A; each injection of the 1 ng/100 μl solution provided 0.02 pmoles of WNT5A; each injection of the 10 ng/100 μl solution provided 0.22 pmoles of WNT5A; each injection of the 100 ng/100 μl solution provided 2.2 pmoles of WNT5A; each injection of the 500 ng/100 μl solution provided 11 pmoles of WNT5A; each injection of the 750 ng/100 μl solution provided 16.5 pmoles of WNT5A, and each injection of the 1000 ng/100 μl solution provided 22 pmoles of WNT5A.

2.3 Assessment of Scarring 70 days after wounding the experimental rats were killed, and the scars resulting from treated wounds and control wounds assessed macroscopically.

The scars of the experimental rats were photographed and assessed using macroscopic scar assessment sheets. Macroscopic assessment of scarring was carried out using a visual analogue scale (VAS) consisting of a 0-10 cm line representing a scale, from left to right, of 0 (corresponding to normal skin) to 10 (indicative of a bad scar). A mark was made by an assessor on the 10 cm line based on an overall assessment of the scar taking into account parameters such as the height, width, contour and colour of the scar. The best scars (typically of small width with colour, height and contour like normal skin) were scored towards the normal skin end of the scale (the left hand side of the VAS line) and bad scars (typically large width, raised with uneven contours and whiter colour) were scored towards the bad scar end of the scale (the right hand side of the VAS line). The marks were measured from the left hand side to provide the final value for the scar assessment in centimetres (to 1 decimal place).

A comparison of the macroscopic VAS scores of scars formed on healing of WNT5A treated wounds and diluent control treated wounds is shown in FIG. 1.

Representative images showing the macroscopic appearance of scars formed on healing of WNT5A treated wounds and diluent control treated wounds are shown in FIG. 2.

### 2.4 Results

Macroscopic analysis of scars formed from incisional wounds (assessed at 70 days post-wounding) showed that the addition of WNT5A to wounds was able to significantly inhibit scarring. Surprisingly, the results show that when WNT5A is administered in an excessive amount the effect of such administration is to increase scarring, over that of diluent control wounds, rather than to inhibit scarring.

Administration of WNT5A at concentrations of 0.5 ng/100 μl, 1 ng/100 μl, 10 ng/100 μl, 100 ng/100 μl and 750 ng/100 μl significantly inhibited scarring, as compared to diluent controls (p<0.05), when assessed using macroscopic VAS. These results, which are shown in FIG. 6, arise as a result of improvements brought about in the abundance and/or orientation of ECM components in the dermis. These microscopic improvements significantly improve the macroscopic appearance of scars formed, such that treated wounds resemble the surrounding un-wounded dermis.

That scarring is effectively inhibited by these concentrations of WNT5A is clearly illustrated in FIG. 7 which shows representative macroscopic images of treated and control treated scars. Scars resulting from wounds treated with therapeutically effective amounts of WNT5A are considerably more difficult to detect than the scars produced on healing of control treated wounds.

In contrast, administration of WNT5A at 1000 ng/100 μl significantly increased scarring when compared to controls (p<0.01). This inhibition of scarring, as noted with reference to macroscopic assessment, is clearly shown in FIGS. 6 and 7. These Figures respectively show scores generated on macroscopic VAS assessment of scars produced from treated and control wounds, and representative macroscopic images illustrating treated scars and control scars.

The results show that a therapeutically effective amount of WNT5A is capable of inhibiting scarring. These results also provide guidance as to how therapeutically effective and non-therapeutically effective amounts of WNT5A may be determined.

### 3. Results Summary

The results reported under headings 1.3 and 2.4 above clearly illustrate that a therapeutically effective amount of WNT5A is capable of inhibiting scarring. These results also provide guidance as to how therapeutically effective and non-therapeutically effective amounts of WNT5A may be determined, as well as providing information as to the amount of WNT5A, or a therapeutically effective fragment or derivative thereof, that may constitute a therapeutically effective amount able to inhibit scarring if provided to a centimetre of wound or scar.

Given the similarities between the biological mechanisms involved in scarring and fibrotic disorders the results reported above also clearly indicate that therapeutically effective amounts of WNT5A are suitable for the prevention and/or treatment of fibrotic disorders.

| Sequence Information | |
|---|---|
| Amino acid sequence of human WNT5A | |
| (Sequence ID No. 1) | |
| 1 | plqksigils pgvalgmags amsskfflva laiffsfaqv vieanswwsl gmnnpvqmse |
| 61 | vyiigaqplc sqlaglsqgq kklchlyqdh mqyigegakt gikecqyqfr hrrwncstvd |
| 121 | ntsvfgrvmq igsretafty avsaagvvna msracregel stcgcsraar pkdlprdwlw |
| 181 | ggcgdnidyg yrfakefvda rererihakg syesarilmn lhnneagrrt vynladvack |
| 241 | chqvsgscsl ktcwlqladf rkvgdalkek ydsaaamrln srgklvqvns rfnspttqdl |
| 301 | vyidpspdyc vrnestgslg tqgrlcnkts egmdgcelmc cgrgydqfkt vqterchckf |
| 361 | hwccyvkckk cteivdqfvc k |

cDNA derived from mRNA sequence of human WNT5A

(Sequence ID No. 2)

| | |
|---|---|
| 1 | agttgcctgc gcgccctcgc cggaccggcg gctccctagt tgcgccccga ccaggccctg |
| 61 | cccttgctgc cggctcgcgc gcgtccgcgc cccctccatt cctgggcgca tcccagctct |
| 121 | gccccaactc gggagtccag gcccgggcgc cagtgcccgc ttcagctccg gttcactgcg |
| 181 | cccgccggac gcgcgccgga ggactccgca gccctgctcc tgaccgtccc cccaggctta |
| 241 | acccggtcgc tccgctcgga ttcctcggct gcgctcgctc gggtggcgac ttcctccccg |
| 301 | cgccccctcc ccctcgccat gaagaagtcc attggaatat taagcccagg agttgctttg |
| 361 | gggatggctg gaagtgcaat gtcttccaag ttcttcctag tggctttggc catatttttc |
| 421 | tccttcgccc aggttgtaat tgaagccaat tcttggtggt cgctaggtat gaataaccct |
| 481 | gttcagatgt cagaagtata tattatagga gcacagcctc tctgcagcca actggcagga |
| 541 | ctttctcaag gacagaagaa actgtgccac ttgtatcagg accacatgca gtacatcgga |
| 601 | gaaggcgcga agacaggcat caaagaatgc cagtatcaat tccgacatcg aaggtggaac |
| 661 | tgcagcactg tggataacac ctctgttttt ggcagggtga tgcagatagg cagccgcgag |
| 721 | acggccttca catacgcggt gagcgcagca ggggtggtga acgccatgag ccgggcgtgc |
| 781 | cgcgagggcg agctgtccac ctgcggctgc agccgcgccg cgcgccccaa ggacctgccg |
| 841 | cgggactggc tctggggcgg ctgcggcgac aacatcgact atggctaccg ctttgccaag |
| 901 | gagttcgtgg acgcccgcga gcgggagcgc atccacgcca agggctccta cgagagtgct |
| 961 | cgcatcctca tgaacctgca caacaacgag gccggccgca ggacggtgta caacctggct |
| 1021 | gatgtggcct gcaagtgcca tggggtgtcc ggctcatgta gcctgaagac atgctggctg |
| 1081 | cagctggcag acttccgcaa ggtgggtgat gccctgaagg agaagtacga cagcgcggcg |
| 1141 | gccatgcggc tcaacagccg gggcaagttg gtacaggtca acagccgctt caactcgccc |
| 1201 | accacacaag acctggtcta catcgacccc agccctgact actgcgtgcg caatgagagc |
| 1261 | accggctcgc tgggcacgca gggccgcctg tgcaacaaga cgtcggaggg catggatggc |
| 1321 | tgcgagctca tgtgctgcgg ccgtggctac gaccagttca agaccgtgca gacggagcgc |
| 1381 | tgccactgca agttccactg gtgctgctac gtcaagtgca agaagtgcac ggagatcgtg |
| 1441 | gaccagtttg tgtgcaagta gtgggtgcca cccagcactc agccccgctc ccaggacccg |
| 1501 | cttatttata gaaagtacag tgattctggt ttttggtttt tagaaatatt ttttattttt |
| 1561 | ccccaagaat tgcaaccgga accatttttt ttcctgttac catctaagaa ctctgtggtt |
| 1621 | tattattaat attataatta ttatttggca ataatggggg tgggaaccaa gaaaaatatt |
| 1681 | tattttgtgg atctttgaaa aggtaataca agacttcttt tgatagtata gaatgaaggg |
| 1741 | gaaataacac ataccctaac ttagctgtgt ggacatggta cacatccaga aggtaaagaa |
| 1801 | atacattttc tttttctcaa atatgccatc atatgggatg ggtaggttcc agttgaaaga |
| 1861 | gggtggtaga aatctattca caattcagct tctatgacca aaatgagttg taaattctct |
| 1921 | ggtgcaagat aaaaggtctt gggaaaacaa aacaaaacaa aacaaacctc ccttccccag |
| 1981 | cagggctgct agcttgcttt ctgcattttc aaaatgataa tttacaatgg aaggacaaga |
| 2041 | atgtcatatt ctcaaggaaa aaaggtatat cacatgtctc attctcctca aatattccat |
| 2101 | ttgcagacag accgtcatat tctaatagct catgaaattt gggcagcagg gaggaaagtc |
| 2161 | cccagaaatt aaaaaattta aaactcttat gtcaagatgt tgatttgaag ctgttataag |
| 2221 | aattaggatt ccagattgta aaaagatccc caaatgattc tggacactag atttttttgt |
| 2281 | ttggggaggt tggcttgaac ataaatgaaa atatcctgtt attttcttag ggatacttgg |
| 2341 | ttagtaaatt ataatagtaa aaataataca tgaatcccat tcacaggttc tcagcccaag |
| 2401 | caacaaggta attgcgtgcc attcagcact gcaccagagc agacaaccta tttgaggaaa |
| 2461 | aacagtgaaa tccaccttcc tcttcacact gagccctctc tgattcctcc gtgttgtgat |
| 2521 | gtgatgctgg ccacgtttcc aaacggcagc tccactgggt cccctttggt tgtaggacag |
| 2581 | gaaatgaaac attaggagct ctgcttggaa aacagttcac tacttaggga tttttgtttc |
| 2641 | ctaaaacttt tattttgagg agcagtagtt ttctatgttt taatgacaga acttggctaa |
| 2701 | tggaattcac agaggtgttg cagcgtatca ctgttatgat cctgtgttta gattatccac |
| 2761 | tcatgcttct cctattgtac tgcaggtgta ccttaaaact gttcccagtg tacttgaaca |
| 2821 | gttgcattta taagggggga aatgtggttt aatggtgcct gatatctcaa agtcttttgt |
| 2881 | acataacata tatatatata tacatatata taaatataaa tataaatata tctcattgca |
| 2941 | gccagtgatt tagatttaca gtttactctg gggttatttc tctgtctaga gcattgttgt |
| 3001 | ccttcactgc agtccagttg ggattattcc aaaagttttt tgagtcttga gcttgggctg |
| 3061 | tggccctgct gtgatcatac cttgagcacg acgaagcaac cttgtttctg aggaagcttg |
| 3121 | agttctgact cactgaaatg cgtgttgggt tgaagatatc ttttttcttt tctgcctcac |
| 3181 | ccctttgtct ccaacctcca tttctgttca ctttgtggag agggcattac ttgttcgtta |
| 3241 | tagacatgga cgttaagaga tattcaaaac tcagaagcat cagcaatgtt tctcttttct |
| 3301 | tagttcattc tgcagaatgg aaacccatgc ctattagaaa tgacagtact tattaattga |
| 3361 | gtccctaagg aatattcagc ccactacata gatagctttt tttttttttt ttttaataag |
| 3421 | gacacctctt tccaaacagt gccatcaaat atgttcttat ctcagactta cgttgtttta |
| 3481 | aaagtttgga aagatacaca tctttcatac cccccttagg caggttggct ttcatatcac |
| 3541 | ctcagccaac tgtggctctt aatttattgc ataatgatat tcacatcccc tcagttgcag |
| 3601 | tgaattgtga gcaaaagatc ttgaaagcaa aaagcactaa ttagtttaaa atgtcacttt |
| 3661 | tttggttttt attatacaaa aaccatgaag tacttttttt atttgctaaa tcagattgtt |
| 3721 | cctttttagt gactcatgtt tatgaagaga gttgagttta acaatcctag cttttaaaag |
| 3781 | aaactattta atgtaaaata ttctacatgt cattcagata ttatgtatat cttctagcct |
| 3841 | ttattctgta cttttaatgt acatatttct gtcttgcgtg atttgtatat ttcactggtt |
| 3901 | taaaaaacaa acatcgaaag gcttatgcca aatggaagat agaatataaa ataaaacgtt |
| 3961 | acttgtatat tggtaagtgg tttcaattgt ccttcagata attcatgtgg agatttttgg |
| 4021 | agaaaccatg acggatagtt taggatgact acatgtcaaa gtaataaaag agtggtgaat |
| 4081 | tttaccaaaa ccaagctatt tggaagcttc aaaaggtttc tatatgtaat ggaacaaaag |
| 4141 | gggaattctc ttttcctata tatgttcctt acaaaaaaaa aaaaaaaaga aatcaagcag |
| 4201 | atggcttaaa gctggttata ggattgctca cattctttta gcattatgca tgtaacttaa |
| 4261 | ttgttttaga gcgtgttgct gttgtaacat cccagagaag aatgaaaagg cacatgcttt |
| 4321 | tatccgtgac cagattttta gtccaaaaaa atgtattttt ttgtgtgttt accactgcaa |
| 4381 | ctattgcacc tctctatttg aatttactgt ggaccatgtg tggtgtctct atgccctttg |
| 4441 | aaagcagttt ttataaaaag aaagcccggg tctgcagaga atgaaaactg gttggaaact |
| 4501 | aaaggttcat tgtgttaagt gcaattaata caagttattg tgcttttcaa aaatgtacac |
| 4561 | ggaaatctgg acagtgctgc acagattgat acattagcct ttgctttttc tctttccgga |
| 4621 | taaccttgta acatattgaa accttttaag gatgccaaga atgcattatt ccacaaaaaa |
| 4681 | acagcagacc aacatataga gtgtttaaaa tagcatttct gggcaaattc aaactcttgt |
| 4741 | ggttctagga ctcacatctg tttcagtttt tcctcagttg tatattgacc agtgttcttt |
| 4801 | attgcaaaaa catatacccg atttagcagt gtcagcgtat tttttcttct catcctggag |
| 4861 | cgtattcaag atcttcccaa tacaagaaaa ttaataaaaa atttatatat aggcagcagc |
| 4921 | aaaagagcca tgttcaaaat agtcattatg ggctcaaata gaaagaagac ttttaagttt |
| 4981 | taatccagtt tatctgttga gttctgtgag ctactgacct cctgagactg gcactgtgta |
| 5041 | agttttagtt gcctacccta gctcttttct cgtacaattt tgccaatacc aagtttcaat |
| 5101 | ttgtttttac aaaacattat tcaagccact agaattatca aatatgacgc tatagcagag |
| 5161 | taaatactct gaataagaga ccggtactag ctaactccaa gagatcgtta gcagcatcag |
| 5221 | tccacaaaca cttagtggcc cacaatatat agagagatag aaaaggtagt tataacttga |
| 5281 | agcatgtatt taatgcaaat aggcacgaag gcacaggtct aaaatactac attgtcactg |
| 5341 | taagctatac ttttaaaata tttatttttt ttaaagtatt ttctagtctt ttctctctct |
| 5401 | gtggaatggt gaaagagaga tgccgtgttt tgaaagtaag atgatgaaat gaatttttaa |
| 5461 | ttcaagaaac attcagaaac ataggaatta aaacttagag aaatgatcta atttccctgt |
| 5521 | tcacacaaac tttacacttt aatctgatga ttggatattt tattttagtg aaacatcatc |
| 5581 | ttgttagcta actttaaaaa atggatgtag aatgattaaa ggttggtatg attttttttt |
| 5641 | aatgtatcag tttgaaccta gaatattgaa ttaaaatgct gtctcagtat tttaaaagca |
| 5701 | aaaaaggaat ggaggaaaat tgcatcttag accattttta tatgcagtgt acaatttgct |
| 5761 | gggctagaaa tgagataaag attatttatt tttgttcata tcttgtactt ttctattaaa |
| 5821 | atcattttat gaaatccaaa aaaaaaaaaa aaaaa |

1. The use of WNT5A, or a therapeutically effective fragment or derivative thereof, in the preparation of a medicament for use in the prevention, reduction or inhibition of scarring.

2. The use according to claim 1, wherein the medicament is for use in the prevention, reduction or inhibition of scarring in a tissue selected from the group consisting of: the skin; the eye; blood vessels; the peripheral or central nervous system; tendons, ligaments or muscle; the oral cavity, lips and palate; the liver; the heart; the brain; digestive tissues; reproductive tissues; the abdominal cavity; the pelvic cavity and the thoracic cavity.

3. The use according to claim 2, wherein the medicament is for use in the prevention, reduction or inhibition of dermal scarring.

4. The use according to claim 2, wherein the medicament is for use in the prevention, reduction or inhibition of scarring in the eye.

5. The use according to claim 1 for the prevention, reduction or inhibition of adhesions, such as those occurring in the abdomen, pelvis or spine; or tendons.

6. The use of WNT5A, or a therapeutically effective fragment or derivative thereof, in the preparation of a medicament for use in the prevention and/or treatment of a fibrotic disorder.

7. The use according to claim 6, wherein the medicament is for use in the prevention and/or treatment of a fibrotic disorder selected from the group consisting of: skin fibrosis; scleroderma; progressive systemic fibrosis; lung fibrosis; muscle fibrosis; kidney fibrosis; glomerulosclerosis; glomerulonephritis; uterine fibrosis; renal fibrosis; cirrhosis of the liver, liver fibrosis; adhesions, such as those occurring in the abdomen, pelvis or spine; chronic obstructive pulmonary disease; fibrosis following myocardial infarction; central nervous system fibrosis, such as fibrosis following stroke; fibrosis associated with neuro-degenerative disorders such as Alzheimer's Disease or multiple sclerosis; fibrosis associated with proliferative vitreoretinopathy (PVR); restenosis; endometriosis; ischemic disease and radiation fibrosis.

8. The use according to any preceding claim, wherein the medicament is an injectable solution comprising WNT5A, or a therapeutically effective fragment or derivative thereof.

9. The use according to any preceding claim, wherein the medicament is a topical medicament comprising WNT5A, or a therapeutically effective fragment or derivative thereof.

10. The use according to any preceding claim, wherein the medicament comprises WNT5A of Sequence ID No. 1.

11. The use according to any preceding claim, wherein the medicament comprises recombinant WNT5A.

12. The use according to any preceding claim, wherein the therapeutically active fragment of WNT5A comprises a palmitoylated fragment of WNT5A.

13. The use according to any preceding claim, wherein the therapeutically active derivative of WNT5A, or a fragment thereof, is a peptoid derivative.

14. The use according to any preceding claim, wherein the WNT5A, or therapeutically active fragment or derivative thereof, is provided in a therapeutically effective amount.

15. The use according to claim 10, wherein the medicament provides less than 2000 ng WNT5A per linear cm of wound, or cm² of a wound or fibrotic disorder, over a 24 hour period.

16. The use according to claim 10, wherein the medicament provides less than 1600 ng WNT5A per linear cm of wound, or cm² of a wound or fibrotic disorder, over a 24 hour period.

17. The use according to any preceding claim, wherein the medicament provides more than 0.2 ng WNT5A per linear cm of wound, or cm² of a wound or fibrotic disorder, over a 24 hour period.

18. The use of WNT5A, or a therapeutically effective fragment or derivative thereof, in the preparation of a medicament for administering between 0.01 and 33 picomoles of WNT5A, or the fragment or derivative, per linear cm of a wound.

19. The use according to claim 18, wherein the medicament is for administering between 0.02 and 16.5 picomoles of WNT5A, or the fragment or derivative, per linear cm of a wound.

20. The use according to claim 18 or claim 19, wherein the medicament is for use in the prevention, reduction or inhibition of scarring.

21. The use of WNT5A, or a therapeutically effective fragment or derivative thereof, in the preparation of a medicament for administering between 0.01 and 33 picomoles of WNT5A, or the fragment or derivative, per cm² of fibrosis associated with a fibrotic disorder.

22. The use according to claim 21, wherein the medicament is for administering between 0.02 and 16.5 picomoles of WNT5A, or the fragment or derivative, per linear cm of a wound.

23. The use according to claim 21 or claim 22, wherein the medicament is for use in the prevention and/or treatment of the fibrotic disorder.

24. The use of WNT5A, or a therapeutically effective fragment or derivative thereof, in the preparation of a medicament for the prevention, reduction or inhibition of scarring, wherein the administration pattern for the medicament comprises administering a therapeutically effective amount of WNT5A, or a fragment or derivative thereof, prior to formation of a wound, and administering a further therapeutically effective amount of WNT5A, or a fragment or derivative thereof, after formation of the wound.

25. A method of preventing, reducing or inhibiting scarring, the method comprising administering a therapeutically effective amount of WNT5A, or a therapeutically effective fragment or derivative thereof, to a patient in need of such prevention, reduction or inhibition.

26. A method of preventing and/or treating a fibrotic disorder, the method comprising administering a therapeutically effective amount of WNT5A, or a therapeutically effective fragment or derivative thereof, to a patient in need of such prevention and/or treatment.

27. A method according to either claim 25 or claim 26, wherein the WNT5A, or therapeutically effective fragment or derivative thereof, is administered by means of a medicament manufactured in accordance with any of claims 1 to 24.
